(19) United States
(12) Patent Application Publication
(10) Pub. No.: US 2026/0234636 A1
(43) Pub. Date: Aug. 13, 2026

(54) ENGINEERING OF piRNA GENERATING SYNTHETIC RNAs FOR GREATER STABILITY AND SUBCELLULAR TARGETING

(71) Applicants: Alex Flynt, Hattiesburg, MS (US); Judith Brown, Tuscon, AZ (US); Olga Mavrodi, Hattiesburg, MS (US); Santosh Poudel, Hattiesburg, MS (US)

(72) Inventors: Alex Flynt, Hattiesburg, MS (US); Judith Brown, Tuscon, AZ (US); Olga Mavrodi, Hattiesburg, MS (US); Santosh Poudel, Hattiesburg, MS (US)

(73) Assignees: University of Southern Mississippi, Hattiesburg, MS (US); Arizona Board of Regents on Behalf of the University of Arizona, Tucson, AZ (US)

(21) Appl. No.: 19/402,618

(22) Filed: Nov. 26, 2025

Related U.S. Application Data
(60) Provisional application No. 63/725,691, filed on Nov. 27, 2024.

(51) Int. Cl. C12N 15/113; A01N 63/60; A01P 7/04; C12N 15/115; C12N 15/82

(52) U.S. Cl. CPC C12N 15/1137; A01N 63/60; A01P 7/04; C12N 15/115; C12N 15/8286; C12N 2310/14; C12N 2310/16; C12N 2310/531

(57) ABSTRACT

The present disclosure relates to synthetic RNAs comprising a piRNA sequence and a target sequence for a target gene of interest, vectors comprising the synthetic RNA, and genetically modified cells comprising the synthetic RNAs. Compositions and kits comprising the same are provided, as well as methods of regulating the expression of a target gene in an organism for pesticide treatment, control, or management. Also, transgenic plants containing the above-mentioned synthetic RNAs are made.

### CROSS REFERENCE TO RELATED APPLICATION

This application claims the benefit of U.S. Provisional Application No. 63/725,691, filed on Nov. 27, 2024, the disclosure of which is hereby incorporated by reference in its entirety as if fully set forth herein.

### INCORPORATION OF MATERIAL OF XML SEQUENCE LISTING BY REFERENCE

The sequence listing submitted herewith as an XML file named “USM1032USSequenceListing” created on Oct. 15, 2025, which is 25,000 bytes in size, is hereby incorporated by reference in its entirety.

### STATEMENT OF GOVERNMENT INTEREST

This invention was made with government support under grant no. 2331378 awarded by the National Science Foundation and Directorate for Technology, Innovation and Partnership. The Government has certain rights in the invention.

### FIELD OF THE DISCLOSURE

This disclosure relates to piwi-interacting RNAs (piRNAs) that can trigger piRNA-driven gene silencing of a target gene's mRNA, as well as compositions comprising the piRNAs. The present disclosure also relates to uses of these piRNAs which are more resistant to enzymatic degradation, to control pest populations in a species-specific manner. The design features of the piRNAs extend the molecule's lifespan and increase efficacy in disrupting essential pest genes to offer a safer, longer lasting and more effective biological approach to pest control.

### BACKGROUND OF THE INVENTION

Agriculture faces significant challenges due to the growing resistance of pests to chemical pesticides. Over 1 billion pounds of pesticides are used annually in the United States, contributing to long-term health risks and environmental damage. Traditional pesticides are becoming less effective as pests evolve, necessitating the development of new, sustainable solutions. RNA interference (RNAi) presents a promising gene-silencing method, capable of controlling pest populations by disrupting vital physiological processes. However, current RNAi technologies are limited by instability and short-lived effects.

RNAi is a promising technology for biopesticide development that is capable of unparallelled specificity and environmental compatibility. Approaches are typically based on double-stranded RNAs (dsRNA), which suffer from limitations such as targeting by gut nucleases, poor stability in planta, and invariant structure. An alternative RNAi mechanism that is independent of double-stranded RNA, the piwi-interacting RNA (piRNA) pathway, may provide an option to enhance gene silencing. This mechanism uses mimicry of endogenously encoded sequences and not structure to trigger production of small RNA effectors.

The commercial potential of piRNA-based biopesticide technology is significant, particularly within the $87 billion global pesticide market, which is growing due to increased food demand and the need for sustainable pest control solutions.

WO2023015319 relates to a synthetic RNA comprising a piRNA sequence and a target sequence for a gene of interest, wherein the synthetic RNA induces silencing of the gene of interest.

Shamimuzzaman, Md et al., “Genome-wide profiling of piRNAs in the whitefly Bemisia tabaci reveals cluster distribution and association with begomovirus transmission”, PLOS ONE, 2019, 14 (3): e0213149, reported that small RNA (sRNA) deep sequencing and genome-wide profiling of piwi-interacting RNAs (piRNAs) in whiteflies fed on tomato yellow leaf curl virus (TYLCV)-infected or non-infected tomato plants caused induction and suppression of a small number of piRNA clusters in whiteflies, suggesting piRNAs may have applications in regulating protein coding genes and in insect-virus interactions (see abstract).

Gainetdinov, I. et al., “Relaxed targeting rules help PIWI proteins silence transposons”, Nature, 2023, 619:394-402, reported that PIWI proteins are better equipped than AGO proteins to target newly acquired or rapidly diverging endogenous transposons without recourse to new small RNA guides.

Priyadarshini, M. et al., “Reprogramming the piRNA pathway for multiplexed and transgenerational gene silencing in C. elegans”, Nat. Methods, 2022, 19 (2): 187-194, reported that piRNAi can induce transgenerational epigenetic silencing of two endogenous genes (him-5 and him-8).

Two classes of small non-coding RNAs known as siRNAs and piRNA play a critical role in gene defense against parasitic genetic elements (i.e. viruses and transposable elements). They differ in function and biogenesis where siRNAs are typically ~18-23 nucleotides long derived from double-stranded RNA precursors through Dicer-mediated cleavage. In insects, siRNAs load into dedicated Argonuate (Ago) proteins where they act as a guide to mRNA targets through complementary base-pairing, which leads to degradation of the target and reduced expression. See Vyas, M. et al., PLOS One 2017, 12, e0168921; Arad, N. et al., Frontiers in Insect Science 2023, 3, 1283334; Malik, H. J. et al., Scientific Reports 2016, 6, 38469; and Zubair, M. et al., Crop Protection 2020, 137, 105308; the disclosures which are each herein incorporated by reference in their entirety.

piRNAs are comparatively longer molecules (~24-32 nucleotides) and are generated in a dicer independent fashion from single-stranded RNA precursors cleaved by Piwi proteins guided by an existing pool of piRNAs. See Mondal, M. et al., Life science alliance 2020, 3; and Shamimuzzaman, M. et al., PLOS One 2019, 14, e0213149; the disclosures of which are each herein incorporated by reference in their entirety. Two main modes of piRNA biogenesis are recognized. During “ping pong” biogenesis partner Piwi proteins load cleaved transcripts produced by the reciprocal partner form new piRNAs. The phasing mechanism is initiated by “responder”-piRNA-directed cleavage of RNA followed by the action of Zuc to convert the transcript into new piRNAs. Regardless of mechanism, piRNAs, like siRNAs, can drive destruction of complementary mRNAs.

Specific sequences, for example, GNRA tetraloops, lend stability to piRNAs due to the non-Watson-Crick secondary structure formation [7]. See Haldar, S. et al., Journal of Chemical Theory and Computation 2015, 11, 3866-3877; the disclosure of which is herein incorporated by reference in its entirety.

A “Mitomer2” aptamer can also be used to direct the piRNAs to the mitochondria where the aptamer binds to mitochondrial membranes which are the site of Zuc localization and piP-bodies [8]. See Tawaraya, Y. et al., Biological and Pharmaceutical Bulletin 2014, 37, 1411-1415, the disclosure of which is herein incorporated by reference in its entirety. These elements give greater stability as well as directing synthetic RNAs to increase entrance of transgene RNAs into the piRNA pathway.

### SUMMARY OF THE INVENTION

The present disclosure provides the incorporation of structural elements into synthetic single-stranded RNAs that can trigger piRNA-driven gene silencing of a target gene's mRNA. These elements give greater stability as well as directing synthetic RNAs to enhance on-target silencing piRNAs.

Specific sequences, for example, “GNRA tetraloops”, lend stability to RNAs due to the non-standard secondary structure formation.

A “Mitomer2” aptamer is used to direct the piRNAs to the mitochondria. The aptamer binds to mitochondrial membranes which are the site of piRNA biogenesis.

In another aspect, the present disclosure provides an improved method for identifying sequences that trigger production of piRNAs though selection of regions where “responder” type piRNAs align. Recognition and cleavage by responder piRNAs is one-step upstream of initiation of phasing biogenesis.

The present invention may be described by the following sentences:

In a first aspect, the present disclosure provides a synthetic RNA for silencing a gene of a target species comprising:
a piwi-interacting RNA (pi-RNA) sequence,
a target sequence targeting the gene of the target species, and
at least one of a)-b):
a) one or more tetraloops appearing at at least one end of the synthetic RNA, and
b) an RNA aptamer that binds to a receptor of a mitochondria of the target species,
wherein the synthetic RNA induces silencing of the gene of the target species.

In the embodiment of ¶[0016], the synthetic RNA is single-stranded.

In any one of ¶¶[0016] to [0017], the synthetic RNA includes one or more of said tetraloops at ends of the synthetic RNA, or two of said tetraloops, one at each of the 3′ and 5′ ends of the synthetic RNA.

In the embodiment of ¶[0018], the one or more tetraloops of the synthetic RNA has the sequence CGCGAGAGCG (SEQ ID NO: 6).

In any one of the embodiments of ¶¶[0016] to [0019], the synthetic RNA includes the RNA aptamer.

In the embodiment of ¶[0020], the RNA aptamer has the sequence CUUAGCCCAUAGCUGGCUGC (SEQ ID NO: 7).

In any one of embodiments ¶¶[0016] to [0021], the target species is a hemipteran organism.

In the embodiment of ¶[0022], the hemipteran organism is a whitefly.

In any one of embodiments ¶¶[0016] to [0023], the target species is Bemisia tabaci.

In the embodiment of ¶[0024], the gene is aquaporin 1 (AQP1) having the amino acid sequence:

(SEQ ID NO. 14)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAGNLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF.

In any one of the embodiments of ¶¶[0016] to [0020], the gene is selected from the group consisting of: aquaporin (AQP1) (SEQ ID NO. 14), alpha glucosidase 1 (AGLU1), vATPase-A, v-ATPase-B, v-APTase-D, v-ATPase-E, Delta-24 sterol reductase (D-24), cholesterol desaturase (C7) (SEQ ID NO. 5), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP=family verprolin-homologous protein (WAVE), or Actin related ⅔.

In any one of embodiments [0016] to [0026], the piwi-interacting RNA is selected from the group consisting of piTop1 (SEQ ID NO. 1), piTop2 (SEQ ID NO. 2), piTop3 (SEQ ID NO. 3), and piTop4 (SEQ ID NO. 4).

In a second aspect, the present disclosure provides a RNA interference (RNAi) construct including synthetic single-stranded RNA that can trigger piRNA-driven gene silencing of a target gene's mRNA as described herein.

In some embodiments of this second aspect, the RNAi construct includes the synthetic RNA for silencing a gene of a target species of any one of ¶¶[0016]-[0027] and a small interfering RNA (siRNA).

In a third aspect, the present disclosure provides a vector comprising the synthetic single-stranded RNA that can trigger piRNA-driven gene silencing of a target gene's mRNA as described herein.

In some embodiments of this third aspect, the vector comprises the synthetic RNA for silencing a gene of a target species of any one of ¶¶[0016]-[0027] or the RNAi construct of ¶[0029].

In the embodiment of ¶[0031], the vector is a transposon vector.

In a fourth aspect, the present disclosure provides a cell comprising a synthetic single-stranded RNA that can trigger piRNA-driven gene silencing of a target gene's mRNA as described herein.

In some embodiments of this fourth aspect, the cell includes the synthetic RNA for silencing a gene of a target species of any one of ¶¶[0016]-[0027], the RNAi construct of ¶[0029], or the vector of any one of ¶¶[0031]-[0032].

In a fifth aspect, the present disclosure provides a composition including a synthetic single-stranded RNA that can trigger piRNA-driven gene silencing of a target gene's mRNA as described herein.

In some embodiments of this fifth aspect, the composition includes the synthetic RNA for silencing a gene of a target species of any one of ¶¶[0016]-[0027], the RNAi construct of ¶[0029], the vector of any one of ¶¶[0031]-[0032], or the cell of ¶[0034].

In a sixth aspect, the present disclosure provides a kit including a synthetic single-stranded RNA that can trigger piRNA-driven gene silencing of a target gene's mRNA, a RNAi construct, a vector, a cell, or a composition as described herein, and instructions for using the synthetic single-stranded RNA that can trigger piRNA-driven gene silencing of a target gene's mRNA as described in any one of the above aspects and embodiments, the RNAi construct as described in any one of the above aspects and embodiments, the vector as described in any one of the above aspects and embodiments, the cell as described in any one of the above aspects and embodiments, or the composition as described in any one of the above aspects and embodiments, for pesticide treatment, pest control, or management of pests.

In some embodiments of this sixth aspect, the kit includes the synthetic RNA for silencing a gene of a target species of any one of ¶¶[0016]-[0027], the RNAi construct of ¶[0029], the vector of any one of ¶¶[0031]-[0032], the cell of ¶[0033], or the composition of ¶[0034], and instructions for using the synthetic RNA as described in any one of the above aspects and embodiments, the RNAi construct as described in any one of the above aspects and embodiments, the vector as described in any one of the above aspects and embodiments, the cell as described in any one of the above aspects and embodiments, or the composition as described in any one of the above aspects and embodiments, for pesticide treatment, control, or management.

In a seventh aspect, the present disclosure provides a method of regulating the expression of a gene in an organism, including step of providing a synthetic single-stranded RNA that can trigger piRNA-driven gene silencing of a target gene's mRNA, a vector, a cell, or a composition as described herein to the organism, wherein the synthetic single-stranded RNA hybridizes to at least a portion of a mRNA transcript of the target gene and causes downregulation of transcription of the target gene.

In some embodiments of this seventh aspect, the method of regulating the expression of a gene in an organism, includes providing the synthetic RNA for silencing a gene of a target species of any one of ¶¶[0016]-[0027], the RNAi construct of ¶[0029], the vector of any one of ¶¶[0031]-[0032], the cell of ¶[0033], or the composition of ¶[0034] to the organism, whereby the synthetic RNA hybridizes to at least a portion of a mRNA transcript of the target gene and causes downregulation of transcription of the target gene.

In the embodiments of any one of ¶¶[0039]-[0040], the organism may be a hemipteran.

In an eighth aspect, the present disclosure provides a transgenic plant comprising the synthetic RNA for silencing a gene of a target species of any one of ¶¶[0016]-[0027], the RNAi construct of ¶[0029], the vector of any one of ¶¶[0031]-[0032], the cell of ¶[0033], or the composition of ¶[0034].

In some embodiments of this eighth aspect, the plant is from the Solanaceae family.

In the embodiments of any one of ¶¶[0042]-[0043], the plant is a tomato.

In a ninth aspect, the present disclosure provides a plant propagation product obtainable from the transgenic plant of any one of ¶¶[0042]-[0044].

### BRIEF DESCRIPTION OF THE DRAWINGS

FIG. 1 shows examples of synthetic RNAs of the present invention.

FIG. 2 shows a diagram from Ozata, D. M., Gainetdinov, I., Zoch, A. et al. PIWI-interacting RNAs: small RNAs with big functions. Nat Rev Genet 20, 89-108 (2019) that includes the Ago3 (Pong) which is seen as the Ago3 loaded piRNA.

FIG. 3 shows phased piRNAs seen downstream of the location at which a responder piRNA binds.

FIG. 4 shows a GNRA tetraloop.

FIG. 5 shows an RNA aptamer (CUUAGCCCAUAGCUGGCUGC-SEQ ID NO. 7) that can bind to the mitochondria.

FIGS. 6A-6C show three constructs targeting six whitefly genes designed for the creation of transgenic tomato plants expressing si- and pi-RNAs for gene expression knockdown. FIG. 6A 35S_Duplex_Cotton, FIG. 6B piTop4_C7 and FIG. 6C piTop4_C7_GNRA_mito2.

FIGS. 7A-7C show survival probability curves over a period of six days for three transgenic lines and the control for three biological replicates (FIGS. 7A, 7B, and 7C). The p-value (p<0.0001) represents significantly lower survival probability for the insects feeding on transgenic tomato lines compared to the control.

FIG. 7D shows that collectively, the survival probability analyses of combined mortality data from all the biological replicates maintained the trend observed in individual replicates.

FIGS. 8A-8D show the qPCR results for AGLU-Rep1 (FIG. 8A), TRE-Rep1 (FIG. 8B), C7-Rep1 (FIG. 8C), and D24-Rep1 (FIG. 8D).

FIGS. 9A-9D show the qPCR results for AGLU-Rep2 (FIG. 9A), TRE-Rep2 (FIG. 9B), C7-Rep2 (FIG. 9C), and D24-Rep2 (FIG. 9D).

FIGS. 10A-10B show the qPCR results for AGLU-Rep1 (FIG. 10A) and TRE-Rep3 (FIG. 10B).

FIGS. 11A-11D show the qPCR results for AGLU-Rep1-3 (FIG. 11A), TRE-Rep1-3 (FIG. 11B), C7-Rep1-3 (FIG. 11C) and D24-Rep3 (FIG. 11D).

FIGS. 12A-12D show that the statistical analyses (single factor ANOVA with post-hoc Tukey's HSD test) of the relative expression knockdowns of the target genes revealed significant down-regulation of expression in whiteflies fed on transgenic tomato lines compared with the control (p<0.05) for AGLU (FIG. 12A), C7 (FIG. 12B), TRE (FIG. 12C), and TRET (FIG. 12D).

### DEFINITIONS

In order to facilitate understanding of the examples provided herein, certain frequently occurring terms are defined herein.

As used herein, “RNAi” refers to a biological process in which RNA molecules inhibit a target RNA transcript expression, typically by causing the destruction of, or sterically blocking the target RNA transcript, resulting in gene silencing or knockdown or gene activation. For example, the target RNA transcript may be an mRNA transcribed from a target gene, wherein the expression of the mRNA and/or target gene are down-regulated by the RNAi agent. In another example, the target transcript may be a non-coding antisense RNA transcript overlapping with a region on a target gene and the expression of the target gene is upregulated by the RNAi agent.

As used herein, “piRNAs”, also known as piwi-interacting RNAs, are a class of short non-coding RNA molecules (usually 26-31 nucleotides long) expressed in cells. They form RNA protein complexes through interaction with piwi proteins. These piRNA protein complexes have been associated with both epigenetic and post-transcriptional gene silencing, especially in the silencing of transposable elements (transposons). Repeat associated small interfering RNA (rasiRNA) is a subspecies of piRNA. The term “piRNA”, as used herein, encompasses all forms of piRNA and derivatives thereof.

As used herein, the terms “trigger” or “piRNA trigger” or “trigger piRNA” or “primary piRNA” are used interchangeably to refer to the piRNA sequence that induces production of secondary piRNAs in a conducive environment. For example, a “piRNA trigger” may be a piRNA sequence that, when placed in an appropriate vector and delivered to a cell or organism, induces production of new piRNAs within the cell or organism. The new piRNAs may be produced by the “ping-pong” mechanism. Alternatively, or in addition, the new piRNAs may be produced by “piRNA phasing”. The new piRNAs are referred to herein as “secondary piRNAs” or “responder piRNAs”.

As used herein, the terms “small interfering RNA” and “siRNA” are used interchangeably to refer to a class of double-stranded non-coding RNA molecules. siRNA operates within the RNA interference (RNAi) pathway by interfering with expression of specific genes by degrading mRNA after transcription, thus preventing translation.

As used herein, the term “gene expression” or linguistic variants thereof refer to the process of converting genetic information encoded in a gene into RNA (e.g., mRNA, IRNA, tRNA. or snRNA) through “transcription” of the gene (i.e., via the enzymatic action of an RNA polymerase), and for protein encoding genes, into proteins through “translation” of mRNA. Gene expression can be regulated at many stages in the process. “Up-regulation” or “activation” refer to regulation that increases and/or enhances the production of gene expression products (e.g., RNA or proteins), while “down-regulation” or “repression” or “silencing” refer to regulation that decreases production. Molecules (e.g., transcription factors) that are involved in up-regulation or down-regulation are often called “activators” and “repressors”, respectively.

As used herein, the term “gene silencing” or “silencing” when used in reference to a gene or gene expression refers to methods for interrupting or suppressing expression of a gene. Gene silencing can occur at the transcriptional or translational level. Gene silencing can indicate a partial suppression of gene expression (e.g. a reduction of gene expression). Alternatively, gene silencing can indicate a complete suppression of gene expression (e.g. an elimination of gene expression).

As used herein, the terms “gene of interest” and “GOI” are used interchangeably to refer to the gene for which modulation of expression is intended. For example, the GOI may be the gene for which silencing of gene expression is desired.

As used herein, the term “hemipteran” refers to an order of insects that share a common arrangement of sucking mouthparts. The defining feature of hemipterans is their “beak” in which the modified mandibles and maxillae form a “stylet” which is sheathed within a modified labium. Hemiptera belong to the insect superorder Paraneoptera. Hemiptera, which includes multiple suborders, including auchenorrhyncha (e.g. cicadas, leafhoppers, treehoppers, planthoppers, froghoppers), coleorrhyncha (e.g. moss bugs), heteroptera (e.g. shield bugs, seeds bugs, assassin bugs, flower bugs, sweetpotato bugs, water bugs), and stemorrhyncha (e.g. aphids, whiteflies, scale insects).

The terms “RNA interference” and “RNAi” as used interchangeably herein to broadly refer to biological processes wherein RNA molecules are involved in sequence-specific gene suppression.

The term “synthetic” when used in reference to nucleic acid molecules (e.g., RNA) refers to non-natural molecules made directly (e.g., in a laboratory) or indirectly (e.g., from expression in a cell of a construct made in a laboratory’) by mankind.

As used herein, the term “transposon” or “transposable element” refers to a DNA sequence that can move and integrate into different locations within the genome. “Transposition” refers to the movement of a transposon. Transposition can create and/or reverse mutations within the genome.

As used herein, the term “tetraloop” refers to a type of four-base loop motif found in hairpin or stem-loop RNA secondary structures that cap duplexes at one end, linking the two strands comprising the duplex, and provide stability to the hairpin structure. Suitable examples of tetraloops may include GNRA (SEQ ID No. 6-CGCGAGAGCG), UNCG, CUUG, ANYA, CUYG, UMAC, CUUG, UNAC.

As used herein, the term “aptamer” refers to oligonucleotides (e.g. short oligonucleotides or deoxyribonucleotides), that bind (e.g. with high affinity and specificity) to proteins, peptides, and small molecules. Aptamers may be RNA. Aptamers may have secondary or tertiary structure and, thus may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLOS ONE 5 (12): e15004). Applying the SELEX and the SOMAmer technology includes for instance adding functional groups that mimic amino acid side chains to expand the aptamer's chemical diversity. As a result, high affinity aptamers for protein may be identified and enriched. Aptamers may exhibit many desirable properties for targeted drug delivery, such as ease of selection and synthesis, high binding affinity and specificity, low immunogenicity, and versatile synthetic accessibility. Preferably, the aptamers employed in the present invention are suitable for binding to mitochondria.

The articles “a” and “an” are used herein to refer to one or to more than one (i.e., to at least one) of the grammatical object of the article.

As used herein, the terms “comprise”, “include”, and linguistic variations thereof denote the presence of recited feature(s), element(s), method step(s), etc. without the exclusion of the presence of additional feature(s), element(s), method step(s), etc.

The term “consisting of” and linguistic variations thereof, denotes the presence of recited feature(s), element(s), method step(s), etc. and excludes any unrecited feature(s), element(s), method step(s), etc., except for ordinarily associated impurities.

The phrase “consisting essentially of” denotes the recited feature(s), element(s), method step(s), etc. and any additional feature(s), element(s), method step(s), etc. that do not materially affect the basic nature of the composition, system, or method.

As used herein, “effective amount” refers to the quantity or concentration of synthetic RNA, RNAi construct, or composition as described herein required to downregulate expression of a target gene of an organism or to produce a phenotypic effect on a cell or an organism such that infection and/or transmission of a disease caused by the cell or the organism is reduced.

As used herein, “expression of a target gene” refers to the transcription and accumulation of the RNA transcript encoded by a target gene and/or translation of the mRNA into protein. The term “down-regulate” refers to any of the methods known in the art by which interfering RNA molecules reduce the level of primary RNA transcripts, mRNA or protein produced from a target gene.

As used herein, “down-regulation” refers to a situation whereby the level of RNA or protein produced from a gene is reduced by at least 10%, by at least 33%, by at least 50%, or by at least 80%. In some embodiments, down-regulation refers to a reduction in the level of RNA or protein produced from a gene by at least 80%, by at least 90%, by at least 95%, or by at least 99%, e.g., within cells of the insect as compared with an appropriate control insect which has for example, not been exposed to the disclosed synthetic RNA or RNAi construct or has been exposed to a control construct. In some embodiments, down-regulation refers to a reduction in RNA or protein levels sufficient to result in a detectable change in a phenotype of the insect as compared with an appropriate insect control, such as reduction of transmission and/or infection capability.

“Identity” as used herein refers to the subunit sequence identity between two polymeric molecules particularly between two nucleotide molecules, such as, between two nucleotide sequences molecules. The identity or extent to which two nucleotide sequences have the same residues at the same positions in an alignment is often expressed as a percentage. The identity between two nucleotide sequences is a direct function of the number of matching or identical positions; e.g., if half (e.g., five positions in a polymer ten nucleotides in length) of the positions in two sequences are identical, the two sequences are 50% identical; if 90% of the positions (e.g., 9 of 10), are matched or identical, the two sequences are 90% identical.

“Homologous” refers to the subunit sequence identity between two polymeric molecules, e.g., between two nucleotide molecules, such as, two DNA molecules or two RNA molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the synthetic RNA, RNAi construct, and/or composition of the invention or be shipped together with a container which contains the the synthetic RNA, RNAi construct, and/or composition. Alternatively, the instructional material may be shipped separately from the container with the intention that the instructional material and the synthetic RNA, RNAi construct, and/or composition be used cooperatively by the recipient.

### DETAILED DESCRIPTION OF THE INVENTION

Agriculture continues to face mounting challenges as pests evolve resistance to conventional chemical pesticides. Over one billion pounds of pesticides are applied each year in the United States, contributing to environmental degradation and long-term health concerns. RNA interference (RNAi) presents a promising alternative, using gene-silencing to control pest populations in a species-specific way. However, most current RNAi products are unstable and degrade quickly in the field. This invention addresses those limitations, offering a safer, longer lasting, and more effective biological approach to pest control.

Arthropod cells generate a variety of small regulatory RNAs that enact RNAi-based gene silencing. Biopesticide technology is typically designed to exploit the small-interfering RNA (siRNA) pathway where precursor double-stranded RNAs are processed into siRNAs that guide repression of complementary mRNAs. The siRNA pathway is an anti-viral response, though a variety of endogenous siRNAs have been annotated. Most species of arthropods express an additional type of small RNA-mediated defense pathway—the piwi-interacting RNA (piRNA) pathway. This mechanism relies on a specific structure to trigger small RNA production, such as double-stranded RNA. Instead, an existing pool of piRNAs recognize complementary transcripts directing their conversion into piRNAs. Endogenous targets of piRNAs are often parasitic elements such as transposons, though some genic transcripts also are subject to regulation. As piRNA mechanisms allow gene targeting based on sequence rather than structure this presents opportunities for nucleic acid engineering. See Quito, K. et al., Abstract (oral presentation) at Entomological Soc. of America-RNAi Symposium, Portland, OR, Nov. 9-12, 2025.

The present disclosure provides an approach to RNAi pesticide strategies that exploits piRNA biogenesis. Synthetic piRNA triggers ingested by adult whiteflies (Bemisia tabaci) not only silence target genes in whiteflies but often result in significant mortality.

The present disclosure also explored the extensible nature of piRNA-based technology where addition of motifs can enable cell uptake, stability, and organelle targeting. As many major pest species exhibit somatic piRNAs further development of the platform may provide an opportunity for applying RNAi biopesticides where challenges using siRNA have limited application.

The present disclosure leverages Piwi-interacting RNAs (piRNAs) to create a new class of synthetic biopesticides with enhanced stability and targeting ability. Stabilizing sequences prevent enzymatic degradation, while an organelle-binding aptamer improves intracellular delivery and precision. These design features extend the molecule's lifespan and increase efficacy in disrupting essential pest genes. Compatible with scalable manufacturing, this piRNA platform represents a next-generation advance in RNAi-based pest management technologies.

This technology offers a sustainable and species-specific solution for agricultural pest control, reducing reliance on harmful chemical pesticides and minimizing effects on non-target organisms.

The present invention is directed to synthetic RNAs suitable for controlling pest populations by gene silencing. Incorporating synthetic RNA constructs into pesticides offers a transformative and sustainable solution for species-specific pest control, while reducing the reliance on harmful chemical pesticides. The present invention provides at least the following features and advantages:
Agricultural pest control, e.g., targeting pest species without affecting non-target organisms.
Environmental sustainability: Reducing the need for chemical pesticides and their harmful effects.
Biotechnology: Improving RNA-based treatments in agricultural and industrial applications.
Gene regulation studies: Expanding research into piRNA functions in pest control.

RNA interference (RNAi) is a candidate next generation biopesticide that is used as an ubiquitous genetic tool to effectively silence gene expression in animals, plants, and fungi (see Flynt, A. S., Pest Manag. Soc., 77:2179-2187 (2021)), and RNAi promises unparalleled specificity and environmental compatibility (see Flynt, A. S. and J. K. Brown, Application of piRNA-Triggered Gene Silencing in the Phloem-Feeding Whitefly Bemisia tabaci B mitotype NA-ME cryptic species, No. 49 Oral Presentation-Symposium 2025 (Abstract)).

In insects, there are three distinct and recognized RNAi pathways: small-interfering RNA (siRNA), microRNA (miRNA), and Piwi-interacting (piRNA). See Flynt, A. S. et al., at 2179. Engineering siRNAs, miRNAs, and piRNAs are well-understood in the art and is described, for example, in Flynt, A. S. et al., at 2182-2185. Insect RNAi typically exploits the siRNA pathway which is not found in other types of animals such as vertebrates and is mediated by arthropod specific Dicer (Dicer 2 or Dcr2) and Argonaute (Argonaute 2 or Ago2) proteins. See Flynt, A. S. et al., at 2179.

The present invention uses synthetic piRNA inducing molecules to protect plants against insects such as the phloem-feeding whitefly pest and plant virus vector, Bemisia tabaci B mitotype NAF-ME cryptic species. It has been demonstrated that piRNA-generating regions of the whitefly genome can be identified and fused to whitefly target gene sequences, which when ingested can silence whitefly genes. Further, when ingested, the piRNA complementary segment of the exogenous RNA is cleaved and converted into additional piRNAs, which can pair with and silence the target mRNA. See Flynt, A. S. and J. K. Brown, No. 49 Oral Presentation-Symposium, 2025 (Abstract). These single-stranded piRNA trigger molecules are expected to accumulate to higher levels, and potentially result in increased gene silencing, compared to dsRNA. Id. Similar synthetic piRNA inducing molecules can be synthesized to silence genes of other species such as other insect species.

Using this gene silencing technology, the synthetic piRNA inducing molecules can be engineered to improve various aspects of the gene silencing molecules. Characteristics that can be engineered for improved stability and performance include length of sequence, addition of stabilizing folds, and inclusion of targeting elements. The present invention provides these types of additions that can be employed for improving performance of such synthetic piRNA inducing molecules.

### Synthetic RNA

One aspect of the present invention involves the incorporation of tetraloop sequences in the synthetic piRNA inducing molecules, for example, GNRA tetraloop sequences (CGCGAGAGCG) (SEQ ID NO: 6), at one or both of the 3′ and 5′ ends of the synthetic RNAs to stabilize the molecules. These tetraloop sequences inhibit exonuclease activity and stabilize the RNA making it more available for endonucleases like piwi proteins. This strategy is expected to be generally applicable for other synthetic single-stranded piRNA inducing molecules.

This strategy, however, does not work for double-stranded RNA (dsRNA) intended for Dicer processing since Dicer processing engages the ends of dsRNA, which ends would be obscured by the presence of these GNRA tetraloops. Thus, the present method is applicable to approaches that use piRNA-based RNAi.

In some embodiments, the present invention also incorporates a mitochondrial binding aptamer in the piRNA inducing molecules, for example, the mitochondria binding aptamer of SEQ ID NO: 7 (CUUAGCCCAUAGCUGGCUGC). This mitochondrial binding aptamer sequence was first reported a decade ago. Incorporation of the sequence will cause the RNA to be trafficked to the mitochondrial membrane. Present on the membrane of the mitochondria is the endonuclease Zuchinni as well as piwi proteins which produce the phasing-type RNAs that are the type of piRNA used to induce gene silencing. Localizing the RNA to the mitochondrial site of piRNA biogenesis promotes the conversion of engineered constructs based on the synthetic piRNA inducing molecules into piRNAs.

This strategy of incorporating a mitochondrial binding aptamer is generally applicable for piRNA inducing molecules of other types. For example, other known mitochondrial binding aptamers may be incorporated into piRNA inducing molecules.

Accordingly, the present invention demonstrates the following advantages over currently existing RNAi which is typically based on double-stranded RNAs (dsRNA):
Increased stability: The piRNA inducing molecules are more stable due to the use of GNRA tetraloop sequences, reducing degradation.
Enhanced targeting: The integration of a mitochondrial-binding aptamer improves subcellular targeting efficiency.
Potential for scale-up: The system is compatible with large-scale production for industrial use.
Environmental safety: Biopesticides offer a safer alternative to traditional chemical pesticides due to their specificity, thereby reducing toxic exposure to humans and wildlife.

This technology offers a unique advantage by providing a more stable, targeted RNAi-based solution for inducing piRNA, with the potential to reduce chemical pesticide use and minimize health and environmental risks.

Unlike other RNAi technologies that rely on dsRNA, these synthetic piRNA-based molecules can be engineered to improve stability and targeting of the molecule. This offers the opportunity to include functionalities that may lead to superior performance and greater biopesticide activity.

The present disclosure provides a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species,
wherein the synthetic RNA induces silencing of the gene of the target species.

In one embodiment, the synthetic RNA is single-stranded.

In any one of the above embodiments, the synthetic RNA may include one or more of said tetraloops at ends of the synthetic RNA, or two of said tetraloops, one at each of the 3′ and 5′ ends of the synthetic RNA.

In the above embodiment, the one or more tetraloops has the sequence CGCGAGAGCG (SEQ ID NO: 6).

In any one of the above embodiments, the RNA aptamer has the sequence

(SEQ ID NO: 7)
CUUAGCCCAUAGCUGGCUGC.

In any one of the above embodiments, the target species includes but is not limited to an insect.

In any one of the above embodiments, the target species includes but is not limited to a hemipteran.

In any one of the above embodiments, the target species is Bemisia tabaci.

In any one of the above embodiments, the gene may be selected from the group consisting of: aquaporin (AQP1) (SEQ ID NO. 14), alpha glucosidase 1 (AGLU1), vATPase-A, v-ATPase-B, v-APTase-D, v-ATPase-E, Delta-24 sterol reductase (D-24), cholesterol desaturase (C7) (SEQ ID NO. 5), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), or Actin related ⅔.

In any one of the above embodiments, the gene may be aquaporin 1 (AQP1) having the amino acid sequence:

(SEQ ID NO. 14)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAG NLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF.

In any one of the above embodiments, the piwi-interacting RNA may be selected from the group consisting of piTop1 (SEQ ID NO. 1), piTop2 (SEQ ID NO. 2), piTop3 (SEQ ID NO. 3), and piTop4 (SEQ ID NO. 4).

In any one of the above embodiments, the synthetic RNA contains a nucleotide sequence that hybridizes under physiologic conditions of a cell to the nucleotide sequence of at least a portion of a genomic sequence to cause down-regulation of transcription at the genomic level, or an mRNA transcript for a gene to be inhibited (i.e., the “target” gene). In some embodiments, the piRNA comprises no more than 1 in 5 base pairs of nucleotide mismatches with respect to the mRNA transcript of the target gene. In some embodiments, the piRNA has at least 90% (e.g, 90%, 91%, 92%, 93%, 94%, 95%, 96%, 97%, 98%, 99%, 100%) sequence identity with the portion of the mRNA transcript of the target gene to which the piRNA hybridizes.

In some embodiments, the synthetic RNA comprises a nucleotide sequence having at least 75% (e.g., 75%, 80%, 85%, 90%, 91%, 92%, 93%, 94%, 95%, 96%, 97%, 98%, 99%, 100%) sequence identity with the nucleotide sequence selected from the sequences described and exemplified in the EXAMPLES below.

### RNAi Construct

In a second aspect, the present disclosure provides an RNA interference (RNAi) construct comprising a synthetic single-stranded RNA that can trigger piRNA-driven gene silencing of a target gene's mRNA as described herein.

In one embodiment of this second aspect, the RNAi construct comprises a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species, and
a small interfering RNA (siRNA).

In any one of the above embodiments, the RNA interference (RNAi) construct induces silencing of the gene of the target species.

In any one of the above embodiments, the synthetic RNA is single-stranded.

In any one of the above embodiments, the synthetic RNA may include one or more of said tetraloops at ends of the synthetic RNA, or two of said tetraloops, one at each of the 3′ and 5′ ends of the synthetic RNA.

In the above embodiment, the one or more tetraloops has the sequence CGCGAGAGCG (SEQ ID NO: 6).

In any one of the above embodiments, the RNA aptamer has the sequence

(SEQ ID NO: 7)
CUUAGCCCAUAGCUGGCUGC.

In any one of the above embodiments, the target species includes but is not limited to an insect.

In any one of the above embodiments, the target species includes but is not limited to a hemipteran.

In any one of the above embodiments, the target species is Bemisia tabaci.

In any one of the above embodiments, the gene may be selected from the group consisting of: aquaporin (AQP1) (SEQ ID NO. 14), alpha glucosidase 1 (AGLU1), vATPase-A, v-ATPase-B, v-APTase-D, v-ATPase-E, Delta-24 sterol reductase (D-24), cholesterol desaturase (C7) (SEQ ID NO. 5), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP=family verprolin-homologous protein (WAVE), or Actin related ⅔.

In any one of the above embodiments, the gene may be aquaporin 1 (AQP1) having the amino acid sequence:

(SEQ ID NO. 14)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAG NLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF.

In any one of the above embodiments, the piwi-interacting RNA may be selected from the group consisting of piTop1 (SEQ ID NO. 1), piTop2 (SEQ ID NO. 2), piTop3 (SEQ ID NO. 3), and piTop4 (SEQ ID NO. 4).

### Vectors

In a third aspect, the present disclosure provides a vector comprising the synthetic single-stranded RNA that can trigger piRNA-driven gene silencing of a target gene's mRNA as described herein.

In one embodiment of this third aspect, the vector comprises a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species
wherein the synthetic RNA induces silencing of the gene of the target species.

In another embodiment of this aspect, the vector comprises a RNAi construct having a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species,
and a small interfering RNA (siRNA).

In the above embodiment, the RNAi construct induces silencing of the gene of the target species.

In any one of the above embodiments, the synthetic RNA is single-stranded.

In any one of the above embodiments, the synthetic RNA may include one or more of said tetraloops at ends of the synthetic RNA, or two of said tetraloops, one at each of the 3′ and 5′ ends of the synthetic RNA.

In the above embodiment, the one or more tetraloops has the sequence CGCGAGAGCG (SEQ ID NO: 6).

In any one of the above embodiments, the RNA aptamer has the sequence

(SEQ ID NO: 7)
CUUAGCCCAUAGCUGGCUGC.

In any one of the above embodiments, the target species includes but is not limited to an insect.

In any one of the above embodiments, the target species includes but is not limited to a hemipteran.

In any one of the above embodiments, the target species includes but is not limited to a whitefly.

In any one of the above embodiments, the target species is Bemisia tabaci.

In any one of the above embodiments, the gene may be selected from the group consisting of: aquaporin (AQP1) (SEQ ID NO. 14), alpha glucosidase 1 (AGLU1), vATPase-A, v-ATPase-B, v-APTase-D, v-ATPase-E, Delta-24 sterol reductase (D-24), cholesterol desaturase (C7) (SEQ ID NO. 5), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), or Actin related ⅔.

In any one of the above embodiments, the gene may be aquaporin 1 (AQP1) having the amino acid sequence:

(SEQ ID NO. 14)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAG NLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF.

In any one of the above embodiments, the piwi-interacting RNA may be selected from the group consisting of piTop1 (SEQ ID NO. 1), piTop2 (SEQ ID NO. 2), piTop3 (SEQ ID NO. 3), and piTop4 (SEQ ID NO. 4).

In any one of the above embodiments, the vector is a transposon vector.

### A Modified Cell

In a fourth aspect, the present disclosure provides a modified cell comprising a synthetic single-stranded RNA that can trigger piRNA-driven gene silencing of a target gene's mRNA as described herein.

In one embodiment of this fourth aspect, the modified cell comprises a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species
wherein the synthetic RNA induces silencing of the gene of the target species.

In another embodiment of this aspect, the modified cell comprises a RNAi construct having a synthetic RNA for silencing a gene of a target species comprising:
a piwi-interacting RNA (pi-RNA) sequence,
a target sequence targeting the gene of the target species, and
at least one of a)-b):
   a) one or more tetraloops appearing at at least one end of the synthetic RNA, and
   b) an RNA aptamer that binds to a receptor of a mitochondria of the target species,
and a small interfering RNA (siRNA), and a small interfering RNA (siRNA).

In the above embodiment, the RNAi construct of the modified cell induces silencing of the gene of the target species.

In another embodiment of this aspect, the modified cell may comprise a vector having the synthetic RNA for silencing a gene of a target species described above, or a RNAi construct as described above. In some embodiments, the vector is a transposon vector.

### Compositions

In a fifth aspect, the present disclosure provides a composition comprising a synthetic single-stranded RNA that can trigger piRNA-driven gene silencing of a target gene's mRNA as described herein.

In one embodiment of this fifth aspect, the compositions of the present disclosure may comprise a synthetic RNA as described herein in any one of the above embodiments, an RNAi construct comprising a synthetic RNA as described herein in any one of the above embodiments, a vector comprising a synthetic RNA as described herein in any one of the above embodiments, or a modified cell as described herein in any one of the above embodiments, in combination with one or more pharmaceutically or physiologically acceptable carriers, diluents or excipients.

The disclosed synthetic RNAs, RNAi constructs, or vectors may also be admixed, encapsulated, conjugated or otherwise associated with other molecules, molecule structures or mixtures of compounds, such as for example, liposomes, polymers, for assisting in uptake, distribution and/or absorption. The RNAs, RNAi constructs, or vector can be provided in formulations also including penetration enhancers, carrier compounds and/or transfection agents.

### Articles of Manufacture and Kits

In a sixth aspect, the present disclosure provides a kit comprising a synthetic RNA, a RNAi construct, a vector, a cell, or a composition as described above, and instructions for pesticide treatment, pest control, or management of pests.

The article of manufacture comprises a container and a label or package insert on or associated with the container. Suitable containers include, for example, bottles, vials, syringes, IV solution bags, etc. The containers may be formed from a variety of materials such as glass or plastic. The container holds the synthetic single-stranded RNA that can trigger piRNA-driven gene silencing of a target gene's mRNA as described in any one of the above aspects and embodiments, the RNAi construct as described herein in any one of the above aspects and embodiments, the vector as described herein in any one of the above aspects and embodiments, the cell as described herein in any one of the above aspects and embodiments, or the composition as described herein in any one of the above aspects and embodiments by itself, or may be combined with another composition effective for pesticide treatment, pest control, or management of pests.

The components of the kit may be provided in any form, e.g., liquid, dried or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include instructions for use, information about the production of the composition, concentration, date of expiration, batch or production site information, and so forth. The containers can include a dosage of the composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device.

### Methods of Use

In a seventh aspect, the present disclosure provides a method of regulating the expression of a gene in an organism, comprising providing a synthetic single-stranded RNA that can trigger piRNA-driven gene silencing of a target gene's mRNA, a vector, a cell, or a composition as described herein to the organism, wherein the synthetic single-stranded RNA hybridizes to at least a portion of a mRNA transcript of the target gene and causes downregulation of transcription of the target gene.

In one embodiment of this seventh aspect, the method of regulating the expression of a gene in an organism, comprises a step of providing a synthetic RNA, a RNAi construct, a vector, a cell, or a composition as described above, to the organism wherein the synthetic RNA hybridizes to at least a portion of a mRNA transcript of the target gene and causes downregulation of transcription of the target gene.

Methods for detecting reductions in RNA or protein levels are well known in the art and include RNA solution hybridization, Northern hybridization, reverse transcription (e.g., quantitative RT-PCR analysis), microarray analysis, antibody binding, enzyme-linked immunosorbent assay (ELISA), and Western blotting. In some embodiments, the down-regulation is a reduction in RNA or protein levels sufficient to result in a detectable change in a phenotype of the insect as compared with an appropriate insect control, such as reduction of transmission and/or infection capability. This down-regulation can thus be measured by phenotypic analysis of the insect using techniques routine in the art.

### Transgenic Plant and Plant Propagation Product

In an eighth aspect, the present disclosure provides for a transgenic plant comprising a synthetic RNA, a RNAi construct, a vector, a cell, or a composition as described above.

In some embodiments of this aspect, the plant is from the Solanaceae family.

In each of these embodiments, the plant is a tomato.

In another aspect, the present disclosure provides a plant propagation product obtainable from the transgenic plant of any one of the embodiments described above.

### EXAMPLES

The following examples were carried out using the piRNA-based constructs piTop1, piTop2, piTop3 and piTop4. These piRNA-based constructs provide the piRNA inducing portion of the molecules.

The sequences of these piRNA constructs are given below.
piTop1, having the nucleotide sequence:

(SEQ ID NO: 1)
ATTTGCGCACATTATCTAGTGTTACTTGAGAGTCC,

piTop2, having the nucleotide sequence:

(SEQ ID NO: 2)
ATTTGCGCACATTATCTAGTGTTACTTGAGAGTCCGGTGCTGATT
CTAGGAACATTTC,

piTop3, having the nucleotide sequence:

(SEQ ID NO: 3)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTC,

piTop4, having the nucleotide sequence:

(SEQ ID NO: 4)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTCTTGGGTTTCTTGGCCACTGGTGACTCTGGCCGTTGCC
TGTCCGTCGGCGAGT.

The synthetic RNAs of the present invention were formed using these piRNA constructs which were flanked to a target gene, for example, cholesterol desaturase (C7), and in some examples, a tetraloop (GNRA) and a mitochondrial binding aptamer (mito2). Cholesterol desaturase has the nucleotide sequence:

(SEQ ID NO: 5)
TCGCCATTTGAGATTTGCTTCACGGGCTGTGGCTCCCATGTTGGG
TCCTCTCCTTCAGCATGATACCAGACAAAAATGAGATCGTTCACT
TCACATGATTTCCAAGTTCGGACTCGCGCAAAGTCTGGAACTTTT
TCAGAATATGGCACGCTGGTGCATTTTCCACTATTCCCATCGAAC
TGCCAGCCGTGGAAAGGACATTCTAAGCAGTCACCTCTGACCACC
CCACCAAGACCCATGTTGGCCCATGG

### Example 1

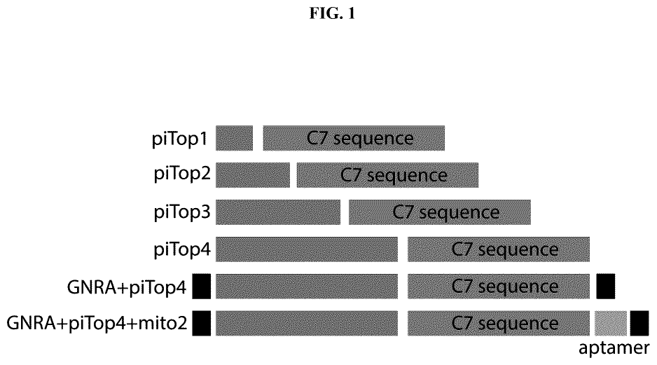

The following exemplified synthetic RNAs are shown in FIG. 1:

piTop1-C7
(SEQ ID NO: 8)
ATTTGCGCACATTATCTAGTGTTACTTGAGAGTCCTCGCCATTTG
AGATTTGCTTCACGGGCTGTGGCTCCCATGTTGGGTCCTCTCCTT
CAGCATGATACCAGACAAAAATGAGATCGTTCACTTCACATGATT
TCCAAGTTCGGACTCGCGCAAAGTCTGGAACTTTTTCAGAATATG
GCACGCTGGTGCATTTTCCACTATTCCCATCGAACTGCCAGCCGT
GGAAAGGACATTCTAAGCAGTCACCTCTGACCACCCCACCAAGAC
CCATGTTGGCCCATGG
piTop2-C7
(SEQ ID NO: 9)
ATTTGCGCACATTATCTAGTGTTACTTGAGAGTCCGGTGCTGATT
CTAGGAACATTTCTCGCCATTTGAGATTTGCTTCACGGGCTGTGG
CTCCCATGTTGGGTCCTCTCCTTCAGCATGATACCAGACAAAAAT
GAGATCGTTCACTTCACATGATTTCCAAGTTCGGACTCGCGCAAA
GTCTGGAACTTTTTCAGAATATGGCACGCTGGTGCATTTTCCACT
ATTCCCATCGAACTGCCAGCCGTGGAAAGGACATTCTAAGCAGTC
ACCTCTGACCACCCCACCAAGACCCATGTTGGCCCATGG
piTop3-C7
(SEQ ID NO: 10)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTCTCGCCATTTGAGATTTGCTTCACGGGCTGTGGCTCCC
ATGTTGGGTCCTCTCCTTCAGCATGATACCAGACAAAAATGAGAT
CGTTCACTTCACATGATTTCCAAGTTCGGACTCGCGCAAAGTCTG
GAACTTTTTCAGAATATGGCACGCTGGTGCATTTTCCACTATTCC
CATCGAACTGCCAGCCGTGGAAAGGACATTCTAAGCAGTCACCTC
TGACCACCCCACCAAGACCCATGTTGGCCCATGG
piTop4-C7
(SEQ ID NO: 11)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTCTTGGGTTTCTTGGCCACTGGTGACTCTGGCCGTTGCC
TGTCCGTCGGCGAGTTCGCCATTTGAGATTTGCTTCACGGGCTGT
GGCTCCCATGTTGGGTCCTCTCCTTCAGCATGATACCAGACAAAA
ATGAGATCGTTCACTTCACATGATTTCCAAGTTCGGACTCGCGCA
AAGTCTGGAACTTTTTCAGAATATGGCACGCTGGTGCATTTTCCA
CTATTCCCATCGAACTGCCAGCCGTGGAAAGGACATTCTAAGCAG
TCACCTCTGACCACCCCACCAAGACCCATGTTGGCCCATGG
GNRA-piTop4-C7-GNRA
(SEQ ID NO: 12)
CGCGAGAGCG (GRNA)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTCTTGGGTTTCTTGGCCACTGGTGACTCTGGCCGTTGCC
TGTCCGTCGGCGAGT (piTop4)
TCGCCATTTGAGATTTGCTTCACGGGCTGTGGCTCCCATGTTGGG
TCCTCTCCTTCAGCATGATACCAGACAAAAATGAGATCGTTCACT
TCACATGATTTCCAAGTTCGGACTCGCGCAAAGTCTGGAACTTTT
TCAGAATATGGCACGCTGGTGCATTTTCCACTATTCCCATCGAAC
TGCCAGCCGTGGAAAGGACATTCTAAGCAGTCACCTCTGACCACC
CCACCAAGACCCATGTTGGCCCATGG (C7)
CGCGAGAGCG (GRNA)
GNRA-piTop4-C7-mito2-GNRA
(SEQ ID NO: 13)
CGCGAGAGCG (GNRA)
ATTTGCGCACATTATCTAGTGTTACTTGAGAGTCCGGTGCTGATT
CTAGGAACATTTCGTTTTTTCTTCTTGGTGCTGGTTTTTTCACTT
CTTTGTTCTTGGGTTTCTTGGCCACTGGTGACTCTGGCCGTTGCC
TGTCCGTCGGCGAGT (piTop4)
TCGCCATTTGAGATTTGCTTCACGGGCTGTGGCTCCCATGTTGGG
TCCTCTCCTTCAGCATGATACCAGACAAAAATGAGATCGTTCACT
TCACATGATTTCCAAGTTCGGACTCGCGCAAAGTCTGGAACTTTT
TCAGAATATGGCACGCTGGTGCATTTTCCACTATTCCCATCGAAC
TGCCAGCCGTGGAAAGGACATTCTAAGCAGTCACCTCTGACCACC
CCACCAAGACCCATGTTGGCCCATGG (C7)
CUUAGCCCAUAGCUGGCUGC (mito2)
CGCGAGAGCG (GNRA)

The constructs were utilized for transient transformation of foliage to cause the biogenesis of piRNAs through exogenous RNAs, using the cholesterol desaturase (C7) sequence as the target gene. The protein C7 was selected as the target gene due to the presence of T residues of ~30 nt in the sequence which makes it a good substrate for phasing. These were cloned directly into pCambia using gene art. The dual piRNA/siRNA constructs underwent cotton transformation which allowed for a comparison of piRNAs vs siRNAs.

Responder piRNA

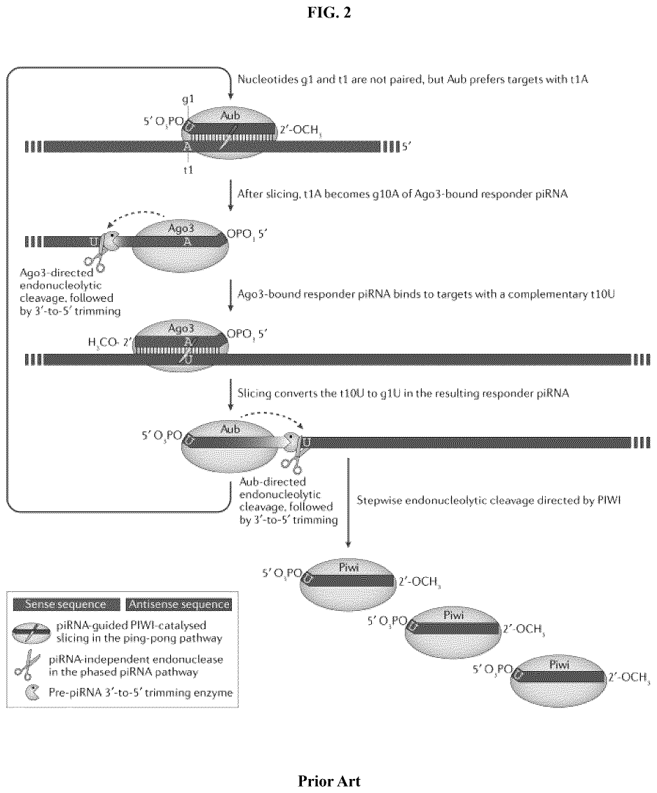

A responder piRNA having a 10 A and 3′U but no 5′1U was identified as Ago3 (Pong) loaded and cleaved by this variety of piRNA which leads to phasing of the transcript. Ago3 loaded piRNA is shown in FIG. 2.

Multiple mechanisms produce piRNAs. Exemplary mechanisms for producing piRNAs include “ping-pong” and “phasing” mechanisms. The ping-pong mechanism of piRNA biogenesis involves piRNAs recognizing their complementary targets, thus causing the recruitment of piwi proteins. piRNAs associate with piwi proteins that have a high frequency of sequence complementarity over 10 nucleotides at their 5′ ends. This sequence complementarity is referred to as the “ping-pong signature”. Association of the piRNAs with piwi proteins results in cleavage of the transcript at a point ten nucleotides from the 5′ end of the primary piRNA, producing the secondary piRNA. These secondary piRNAs are often targeted toward sequences that possess an adenine at the tenth position. The ping pong cycle acts to disrupt gene expression at the transcriptional level.

The “phasing” mechanism of piRNA production involves the targeting and cleavage of a complementary target by a piwi protein associated with piRNA. Once cleaved, the targeted transcript is then processed by additional enzymes (e.g. endonucleases), which leads to the loading of the piwi protein with sequential fragments of the targeted transcript. In this way, the piRNA sequence cleaves a complementary target at periodic intervals (e.g. intervals of approximately 27 nucleotides in length) that are sequentially loaded into the piwi protein. Once loaded with piRNA, the piwi protein enters the germ cell nucleus to co-transcriptionally silence nascent complementary transcripts.

Although the ping-pong and phasing mechanisms are different, a unifying principle is that pre-existing piRNAs convert other cellular RNAs into new piRNAs. When this happens piRNAs bind through complementary base pairing to the target RNAs, which activates cleavage of the target and recruitment of the newly produced RNA fragments into the pathway. Cleavage patterns characteristic of piRNAs can be identified using analysis of high-throughput sequencing.

In some embodiments, methods described herein use existing piRNAs to drive gene silencing of target genes through the generation of on-target piRNAs. This may be performed by feeding animals synthetic RNAs that induce gene silencing via piRNA production. In some embodiments, the synthetic RNAs are designed by fusing curated piRNA sequences to the target sequence. By formulating the curated piRNA, sequence-specific piRNA modes (e.g., ping-pong or phasing) can be invoked. The result is a molecule that after ingestion triggers gene silencing via a mechanism distinct from classic dsRNA approaches. Such methods enable single-stranded RNA-based gene silencing.

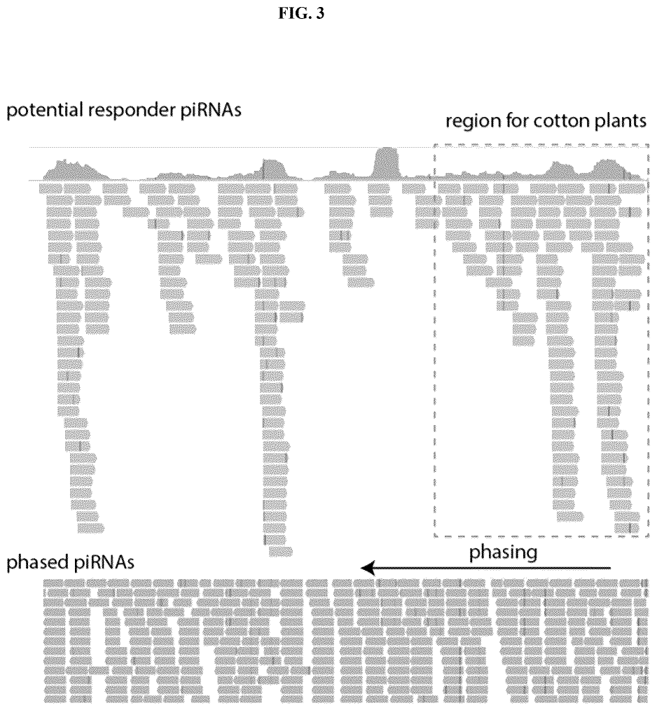

Several piRNAs having strong signals in the piTop locus sequence were chosen as responder piRNAs for cotton transformation. This approach builds on the high confidence sequence used for cotton. The first set of constructs truncate the sequence. Four sequences were developed by removing bases in a left to right manner of the diagram below. The resultant constructs are referred to herein as piTop 1, piTop 2, piTop 3 and piTop 4. The number refers to the number of phased piRNAs seen downstream of the location at which the responder piRNA binds, as shown in FIG. 3.

### GNRA Tetraloop

Another element that was tested was the inclusion of GNRA tetraloops on the ends of the piTop4 sequence. These GNRA tetraloops inhibit exonuclease activity and stabilizes the piTop4 RNA making it more available for endonucleases like piwi proteins.

The sequence of the GNRA tetraloops was SEQ ID NO. 6 (CGCGAGAGCG) (Susanta Haldar, Petra Kührová, Pavel Banáš, Vojtěch Spiwok, Jiří Šponer, Pavel Hobza, and Michal Otyepka, “Insights into Stability and Folding of GNRA and UNCG Tetraloops Revealed by Microsecond Molecular Dynamics and Well-Tempered Metadynamics,” Journal of Chemical Theory and Computation 2015 11 (8), 3866-3877. DOI: 10.1021/acs.jctc.5b00010.

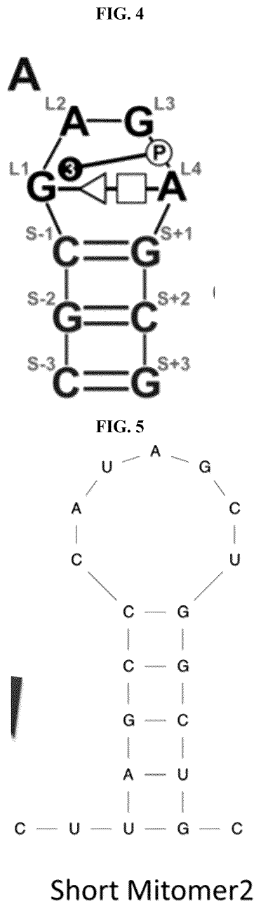

In some embodiments, the synthetic RNA comprises a GNRA tetraloop on at least one of the 5′ end and the 3′ end of the synthetic RNA as shown in FIG. 4. In some embodiments, the GNRA tetraloop is present on both the 5′ end and the 3′ end of the synthetic RNA. The GNRA provides protection to the ends of the synthetic RNA from non-specific cleavage by generic turnover enzymes

### Mitochondria Binding Aptamer

Also tested was the addition of mitochondria binding aptamer. Phasing occurs on the surface of mitochondrial membranes. The mitochondria binding aptamer directs the synthetic RNA to the mitochondria, which enhances the production of piRNAs.

FIG. 5 shows an RNA aptamer that can bind to the mitochondria, namely, (CUUAGCCCAUAGCUGGCUGC-SEQ ID NO. 7) (Tawaraya Y, Hyodo M, Ara M N, Yamada Y, Harashima H., “RNA aptamers for targeting mitochondria using a mitochondria-based SELEX method,” Biol Pharm Bull. 2014; 37 (8): 1411-5. doi: 10.1248/bpb.b14-00112. PMID: 25087963).

EXAMPLES 2 to 4 involve Development of transgenic tomato plants expressing RNA interference (RNAi) constructs designed to silence essential genes in the whitefly Bemisia tabaci (Genn.) cryptic species NAFME, aka ‘B biotype’ [hereafter, B biotype/NAFME cryptic species].

### Rationale:

To use RNAi induced by multiple mechanisms to develop a hybrid double-stranded and single-stranded (ds/ss) RNA biopesticide to reduce whitefly survival, to abate damage due to whitefly feeding (pest) and reduce rates of transmission of plant viruses (vector) and mitigate collective damage associated with this pest and vector.

### Objectives

Develop RNAi constructs that trigger two gene silencing modes (small-interfering RNA (siRNA)+piwi-interacting RNA (piRNA)) to improve RNAi efficiency in whiteflies ingesting transgenic material.
Target previously validated genes (sucrose feeding bioassays-Brown Lab, UA) involved in metabolism [AGLU (alpha glucosidase-1), TRE (trehalase-1), TRET (trehalose transporter-1)] (see Raza, V. M., et al., PLOS One, 2017, 12, e0168921; Arad, N. et al., Frontiers in Insect Science, 2023, 3, 1283334; and Mondal, M. et al., Life Science Alliance, 2020, 3; the disclosures of which are each herein incorporated by reference in their entirety), cholesterol synthesis [D24 (delta 24-sterol reductase), C7 (cholesterol 7-desaturase)], and transcriptional regulation [Crc (cryptocephal)]. Initial experiments were conducted with C7.
Test efficacy of GNRA tetraloops and determined if the modification lends stability to piRNAs based on non-Watson-crick base pairing structure formation to mitigate exonuclease activity.
Test efficacy of “Mitomer2”, whose design incorporated specific sequences that directed piRNAs to the mitochondria, such that the aptamer binds to mitochondrial membrane where the phasing piRNA biogenesis factor Zucchini (Zuc) is localized as well as piP-bodies.
Test efficacy of constructs expressed in transgenic tomato plants, based on whitefly mortality and knockdown of gene expression.

### Experimental Process

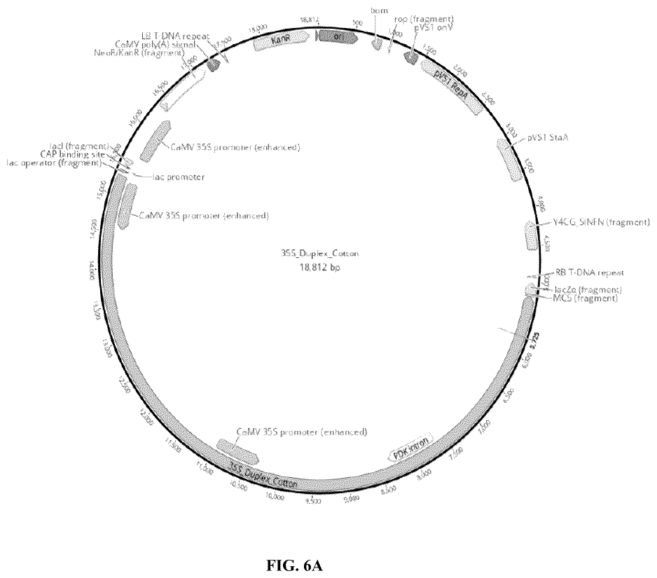

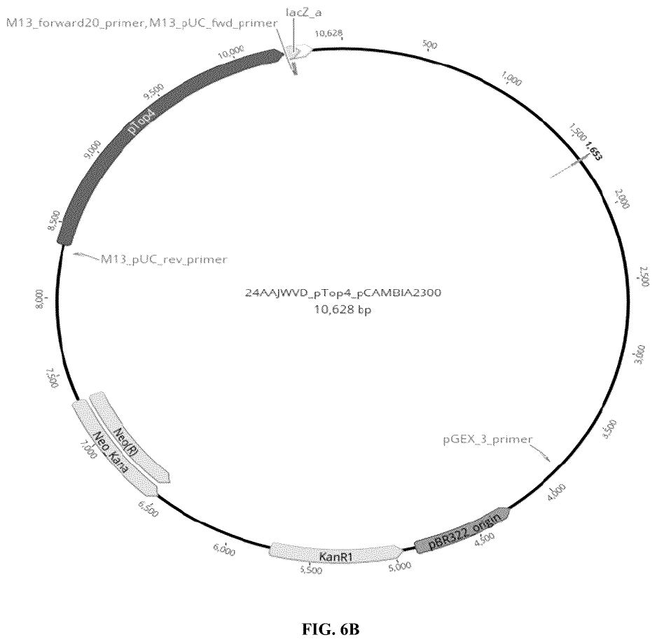

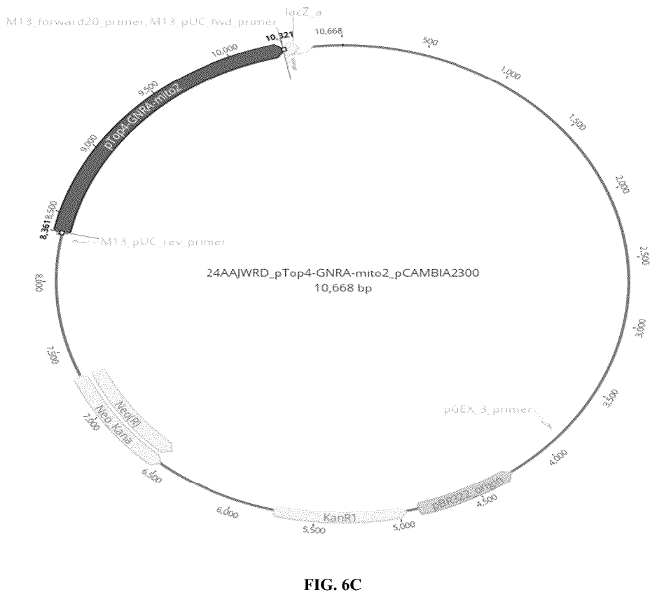

Construct design and transformation of tomato ‘Moneymaker’ (FIGS. 6a-6c).
PCR and sequencing confirmation of transgene cassette integration with specific primers designed to flank the nptII, 35S CaMV promoter, and C7 gene sequence, respectively.
Whitefly feeding bioassay to quantify mortality and knockdown of gene expression based on real-time quantitative PCR amplification.
RNA sequencing small and large (m) RNAs in whiteflies 144 hrs (6 days) and 96 hrs (4 days) post-ingestion-access period (IAP) replicated IAP experiments, respectively. One plant sample to evaluate (in planta), to evaluate efficacy of tomato plant TO progeny expressing si/pi-RNA to cause knock down effect.
Grow plants to fruiting stage, harvest mature fruits, and collect T0 seeds.
Determine transgene copy number in transgenic tomato lines/plants and increase seed from desired events (usually, single copy events).
Increase progeny in subsequent generations (T1-T3) for stability and subsequent whitefly inoculations.

### Example 2

### Background

The objective of this example was to evaluate the efficiency of si- and pi-RNA knockdown in transgenic tomato plants of target genes of interest for three transgenic tomato lines expressing RNAi constructs, post-IAP by adult B. tabaci whiteflies. Mortality was recorded over 6 consecutive days of feeding and Kaplan-Meier survival probability analysis was used to analyze the results for each replicate (separately) and for combined data from three independent biological replicates, and on non-transgenic (control) tomato plants, indicating the effectiveness of the RNAi constructs.

The second objective was to quantify gene expression to determine the extent of gene knockdown in adult whiteflies given ingestion-access periods (IAP) of feeding on tomato plants expressing the genes/sequences of interest, compared to negative control tomato plants.

Real-time quantitative PCR (qPCR) was carried out with cDNA prepared from RNA isolated from whiteflies collected on day-4 and day-6 post-IAP. The results are presented and discussed based on three biological replicates of B. tabaci whitefly ingestion-access feeding experiments with transgenic tomato plants and tomato plants transformed with the respective cassette minus the transgene sequence, as the negative experimental control.

### Methods

### Whitefly Bioassay Ingestion-Access Period (IAP) Bioassay

Adult whiteflies were reared on cotton plants held in a mesh cage that were maintained in an insect-free growth room under LED lights (12:12 day-night cycle) at 79-83° F. Whiteflies were transferred to tomato plants (30 whiteflies/plant) using a hand-held aspirator and allowed 96-hr (4 days) and 144-hr (6 days) ingestion-access periods (IAPs) on transgenic tomato lines expressing the different RNAi constructs. The negative control consisted of tomato plants transformed with the pCAMBIA2300 plasmid that contained no transgene. Three independent biological replicates were performed for each transgenic line and control. Adult whiteflies (n=300±10) were released onto three weeks old plants and were maintained in insect-proof cages under controlled conditions [25±2° C.; 16:8 h (light/dark) photoperiod]. Mortality was recorded daily for six consecutive days, and the data was analyzed statistically.

Sample Collection, RNA Isolation and cDNA Synthesis

Whitefly samples were collected from each transgenic line and control at day-4 and day-6 post-ingestion access period for RNA isolation and molecular analysis. Whiteflies were flash-frozen in liquid nitrogen and stored at −80° C. until RNA extraction. Total RNA was isolated from pooled whiteflies using the RNAzol™ reagent (Sigma-Alrich, USA) following the manufacturer's instructions. The concentration and purity of RNA were determined spectrophotometrically using a NanoDrop™ 2000 (Thermo Fisher Scientific), and RNA integrity was assessed by agarose gel electrophoresis. About 1 μg of total RNA was used for cDNA synthesis using the RevertAid First Strand cDNA Synthesis Kit (Thermo Fisher Scientific) using oligo (dT) primers as per manufacturer's instructions. The cDNA was diluted 10-fold and was used as a template for real-time quantitative PCR analysis.

### Real-Time Quantitative PCR Amplification

The gene expression analysis was performed using real-time quantitative PCR amplification (qPCR) (TaqMan chemistry) on a BioRad CFX96 Touch Real-Time PCR System. Primer pairs were designed to flank the target region for each construct, and for the 18S rRNA gene, the housekeeping reference gene was used for expression normalization. Each reaction was carried out in a 20 μl volume containing 10 μl of PerfeCTa™ qPCR SuperMix (Quantabio), 1 μl of each primer (10 μM) and 0.5 μl of probe, 2 μl of diluted cDNA and nuclease-free water to a final volume of 25 μl. The cycling conditions were as follows: 95° C. for 2 min, followed by 40 cycles of 95° C. for 15 seconds and 55° C. for 30 seconds. All the reactions were conducted in triplicate.

### Statistical Data Analysis

To estimate the survival probabilities of the whiteflies feeding on transgenic and control tomato plants, mortality data from all the replicates were statistically analyzed using Kaplan-Meier survival analysis. The Mantel-Cox log-rank test was applied to assess the significance of differences in mortality among different treatments (transgenic vs control). The relative gene expression levels were calculated using the ΔΔCt program (Applied Biosystems StepOnePlus instrument, Applied Biosystems, USA) method after normalization with the internal control (18S) gene. The extent of expression knockdown in whiteflies fed on transgenic tomato plants was determined by comparing the fold change in expression relative to the control.

### Whitefly Mortality and Gene Knockdown of Transgenes Expressed in Transgenic Tomato Plants

### Results: Whitefly Mortality

Enhanced whitefly mortality was observed in the transgenic tomato lines for the three replicated experiments, compared to the negative transformed control (plasmid without transgene). In the first replicate, the Duplex transgenic line exhibited the highest mortality (~72%), followed by the piTop4_C7_GNRA_mito2 (~58%) transgenic line and piTop4_C7 (~44%) transgenic line, whereas the control group recorded only ~15% mortality. A similar trend was observed in the second replicate, where mortality reached approximately 79% in the Duplex, 71% in piTop4_C7_GNRA_mito2, and 65% in piTop4_C7, while the control maintained a lower (natural) mortality rate (~13%).

Whiteflies given a 144-hour IAP on the transgenic lines 35S_Duplex_cotton, piTop4_C7, and piTop4_C7_GNRA_mito2 showed ~76%, ~61%, and ~69% mortality, respectively, in the third replicate, while the control remained at 14%. The results demonstrated reproducible and significantly higher mortality rate in whiteflies feeding on transgenic tomato lines compared to the control plants, indicating the efficacy of the transgene constructs.

### Example 3

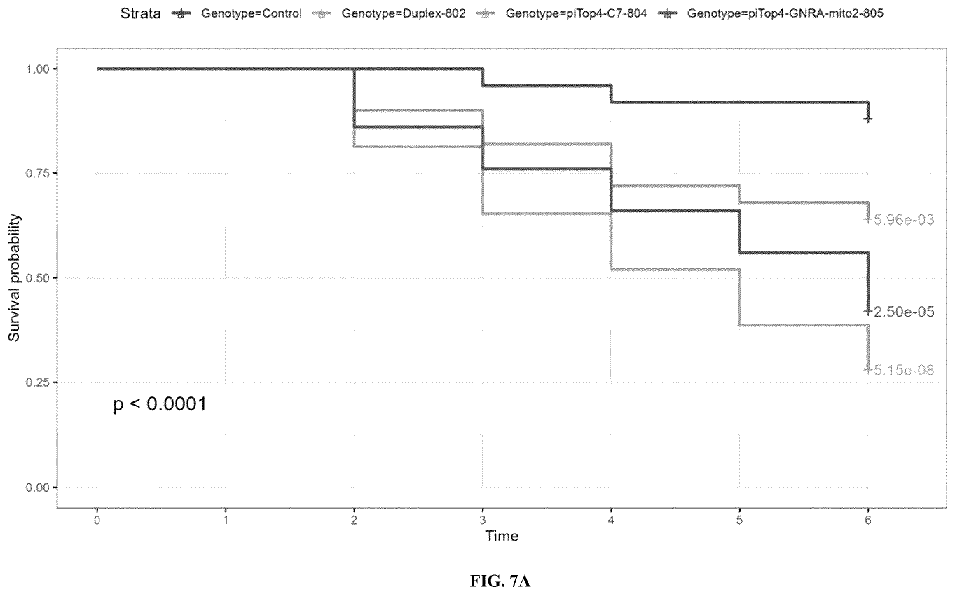

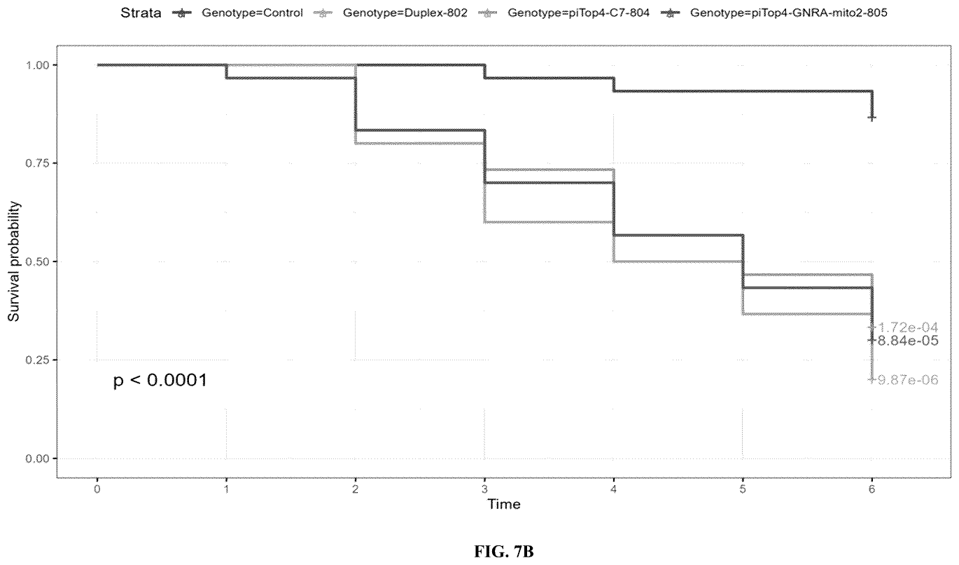

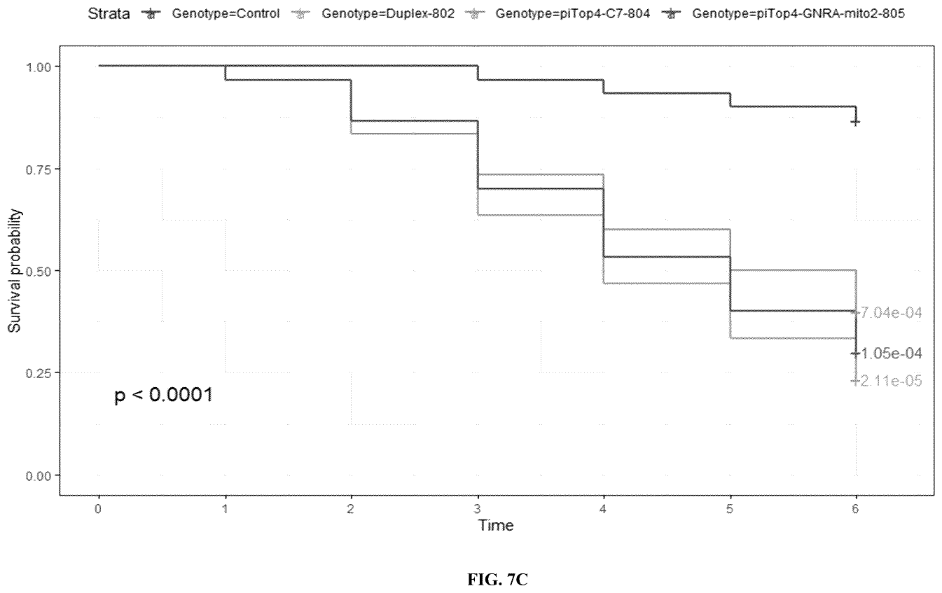

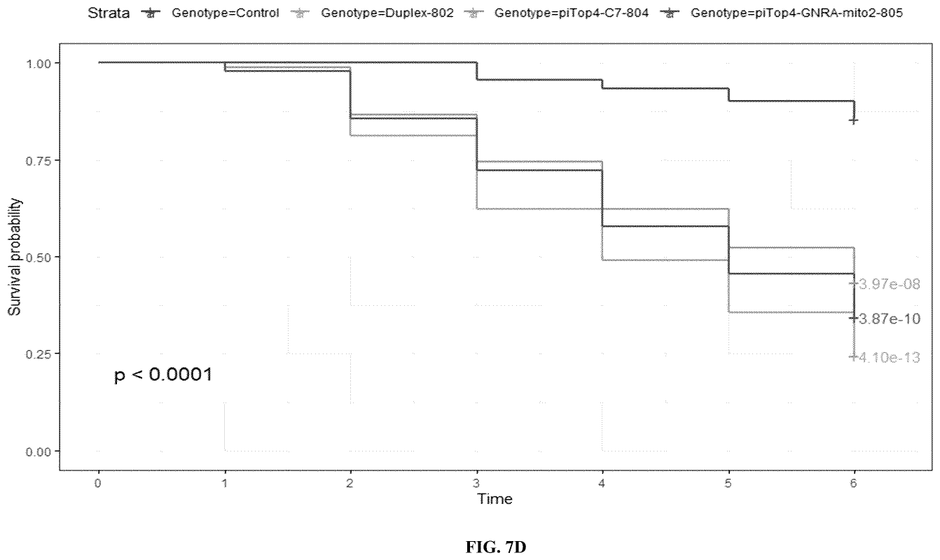

To evaluate the efficacy of RNAi constructs expressed in transgenic tomato lines, whitefly ingestion (feeding) bioassays were conducted for three independent transgenic lines (Line-802, Line-804, and Line-805) and the negative control, for three biological replicates, each. Mortality was recorded daily. The Kaplan-Meier (K-M) survival analysis (FIGS. 7A-7C) revealed that across all three replicates there was a significant reduction in insect survival in all transgenic lines when compared with the control (p<0.0001). The K-M survival probability curve showed distinct clustering between transgenic and control treatments, emphasizing measurable effects of RNAi on whitefly survival. The results showed that whitefly mortality was consistent and significantly higher for those given a 144-hr IAP on transgenic tomato lines expressing the construct of interest, compared to the whiteflies allowed identical IAPs on negative control transgenic tomato plants, minus the construct of interest.

The Kaplan-Meier survival analyses of whitefly feeding on transgenic tomato lines are shown in FIGS. 7A-7D. The p-value (p<0.0001) represents significantly lower survival probability for the insects feeding on transgenic tomato lines compared to the control.

### Example 4

### Differential Gene Expression and Knockdown in Adult Whiteflies

To determine whether adult whitefly mortality was correlated with gene expression knockdown, cDNA was prepared from the total RNA (1 μg) previously isolated from whiteflies, IAP on each transgenic line as well as the control, and real-time qPCR was performed for six target genes, associated with essential physiological processes. In all three replicates, adult whiteflies given a 96-hr IAP on transgenic tomato lines exhibited significant and variable suppression of expression of the target genes compared with those fed on the control plants.

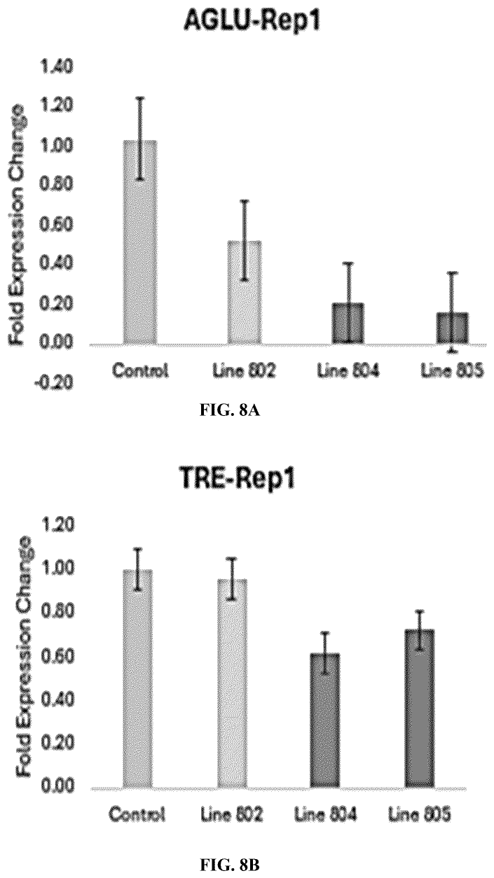

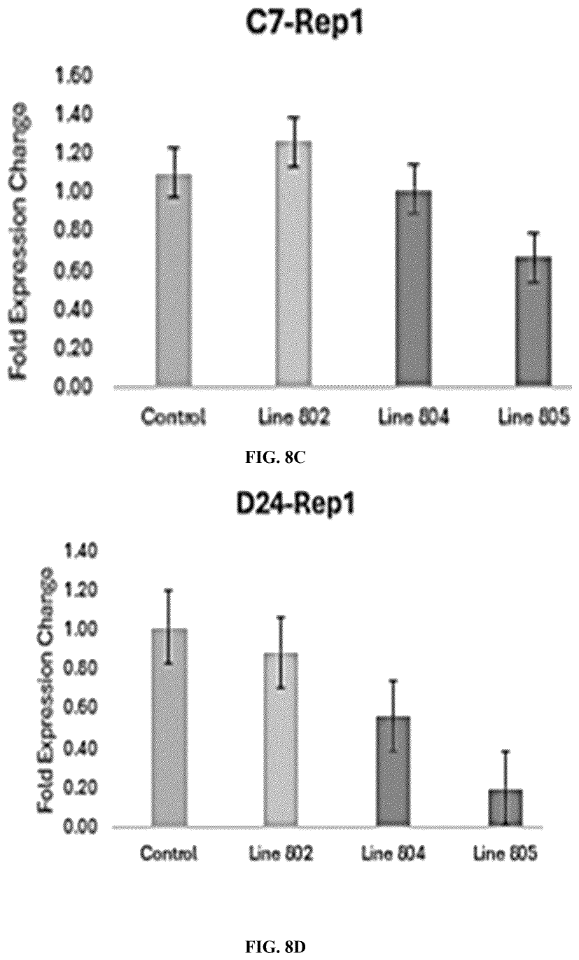

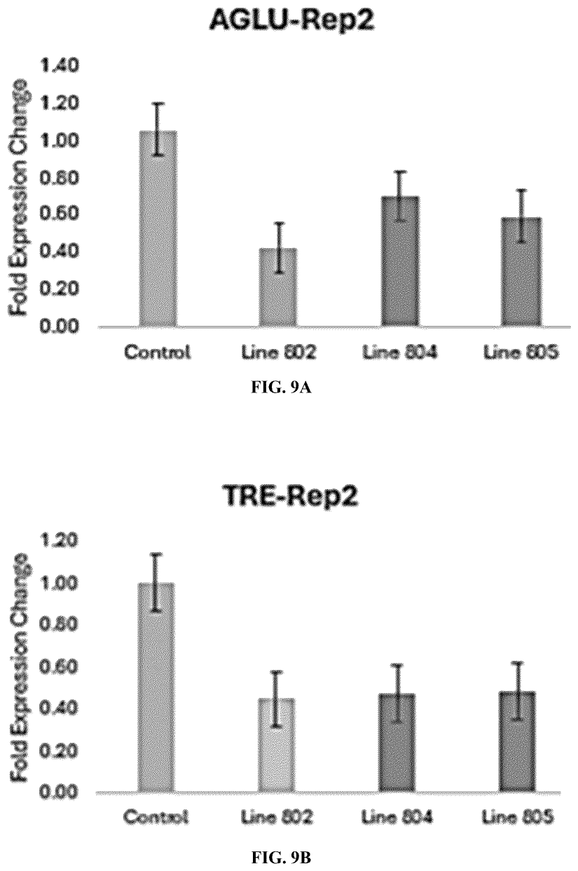

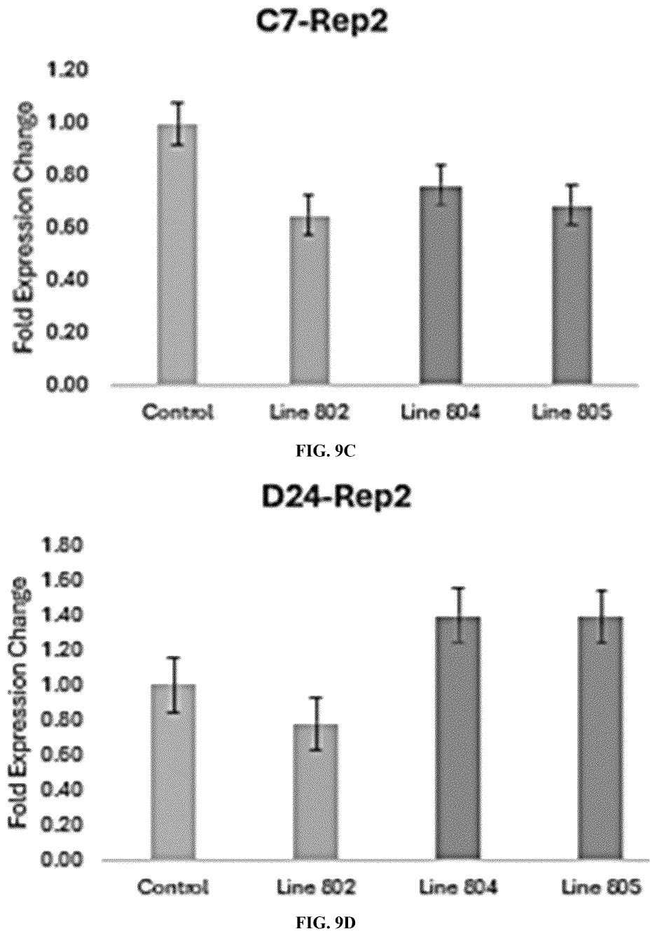

The qPCR analysis showed that the piTop4_C7_GNRA_mito2 transgenic line exhibited the maximum overall suppression across the target genes, although the degree of downregulation varied among individual genes, followed by the piTop4_C7 transgenic line and the 35S_Duplex_cotton transgenic line (FIGS. 8A-8D). The qPCR results from the second replicate also showed a similar trend where the piTop4_C7_GNRA_mito2 showed the strongest gene suppression, while 35S_Duplex_cotton and piTop4_C7 displayed moderate knockdown levels except for the AGLU where a comparatively greater level of suppression was observed (FIGS. 9A-9D).

Interestingly, expression of D24 in piTop4_C7 and piTop4_C7_GNRA_mito2 lines during replicate 2 was unusually higher than that of the control and Duplex lines. In the third replicate, piTop4_C7_GNRA_mito2 and 35S_Duplex_cotton followed a similar trend to previous biological replicates, except for D24, which showed significant downregulation. The piTop4_C7 line demonstrated moderate suppression for most genes, except for Crc, which showed highly significant reduced gene expression in replicate-3.

Real time qPCR results showed a variable gene expression down regulation ranging from ~25% to 80% with an overall average of ~50% reduced gene expression of the target genes in whiteflies given a 144-hr IAP on transgenic tomato plants, confirming RNAi mediated knockdown. The consistency of results across three biological replicates demonstrated the reproducibility of the feeding bioassay and its corresponding phenotypic impact on whitefly survival.

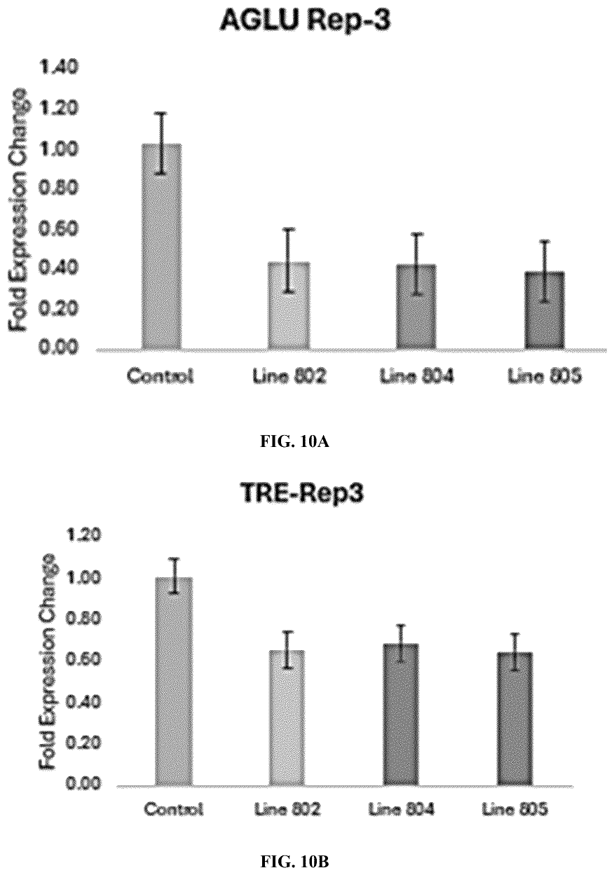

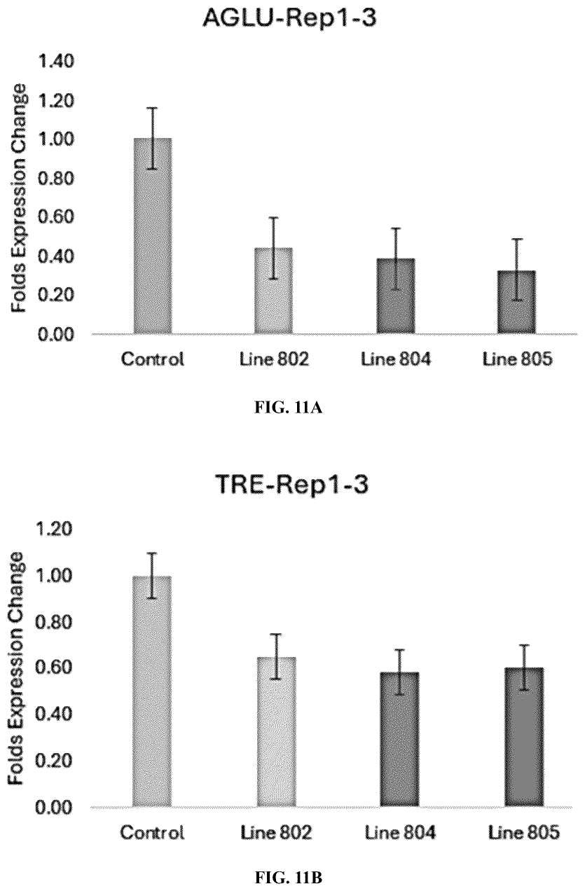

The relative expression of target genes in whiteflies fed on transgenic tomato lines showing significant fold expression reduction in target genes compared with control are shown in FIGS. 8A-8D, 9A-9D, 10A-10B, and 11A-11D. The data shows that individual replicates (in FIGS. 8A-8D, 9A-9D, and 10A-10B) as well as combined analyses of all biological replicates (in FIG. 11A-11D) demonstrated an overall suppression trend.

### Statistical Analysis of the Relative Expression Knockdown:

The statistical analysis of relative gene expression data showed significant differences in expression across the whiteflies fed on transgenic lines compared with the control (FIGS. 8A-8D, 9A-9D, 10A-10B, and 11A-11D). Whitefly samples collected from three transgenic lines revealed significantly reduced levels of alpha glucosidase 1 (AGLU) compared with the control which exhibited comparatively higher expression levels.

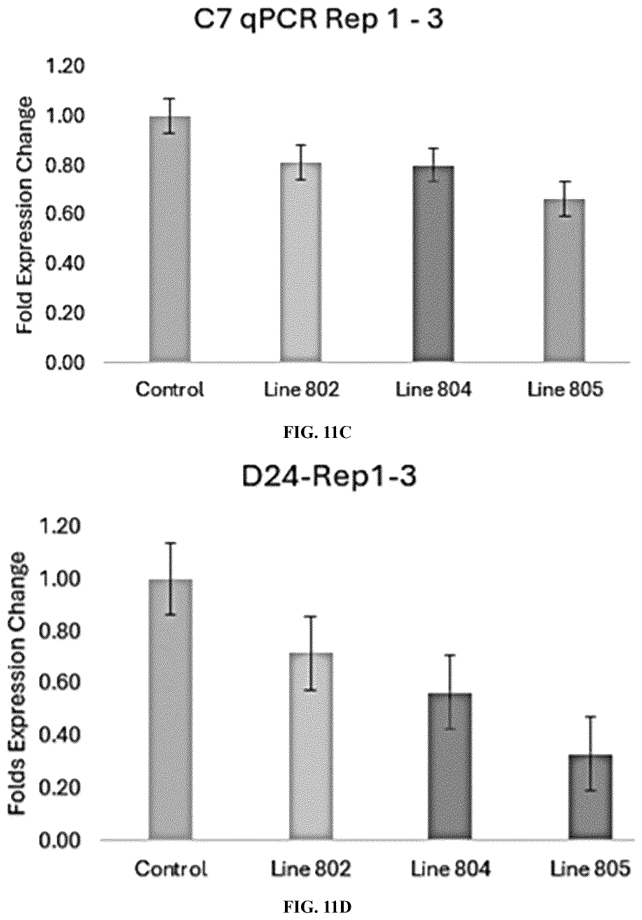

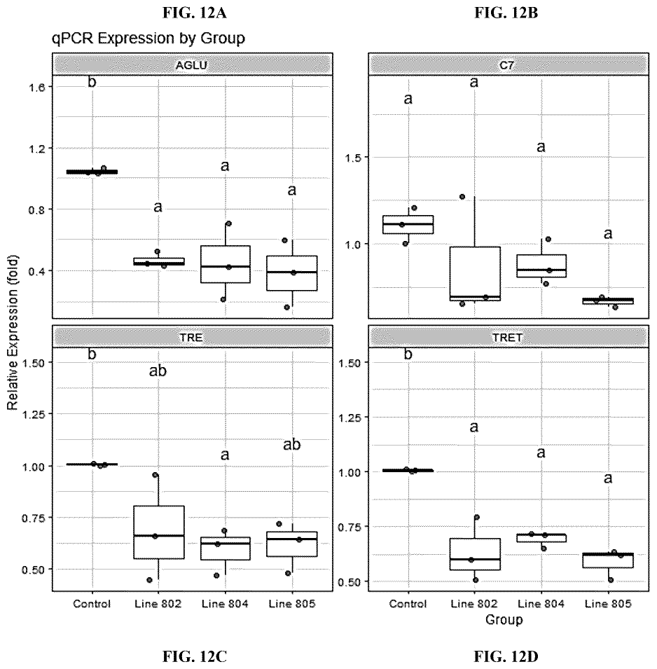

A different trend was observed for C7 where expression levels in whiteflies were collected from line-804. Although lower than the control, C7 expression remained relatively higher compared to the other two transgenic lines (802 and 805) which demonstrated a significant reduction in C7 expression. The expression levels of D24 were greatest in the control while intermediate expression levels were observed in lines 802 and 804 with the lowest in whiteflies fed on transgenic line-805, showing a progressive suppression of expression. For trehalase 1 (TRE), the whiteflies fed on control plants exhibited higher expression levels, while line-802 showed intermediate suppression compared to the other two transgenic lines 804 and 805 where the expression was further downregulated. The expression levels of trehalose transporter-1 (TRET) were significantly reduced in whiteflies fed on all three transgenic lines compared with the control, with no significant variation among the samples collected from transgenic lines. Overall, these results demonstrate a consistent expression knockdown of the targeted genes relative to the control, with transgenic line-805 demonstrating the strongest suppression across multiple targets (FIGS. 12A-12D).

### Correlation Between Mortality and Gene Suppression

The 35S_Duplex_Cotton transgenic line showed the highest mortality, and the piTop4_C7_GNRA_mito2 transgenic line consistently resulted in the most robust reduced target gene expression. The observed inverse relationship between gene expression and survival probability supports the effectiveness of RNAi constructs in silencing important insect genes. Gene knockdown alone may not fully explain the resultant mortality. For example, differences in expression levels of si/pi-RNA and/or whitefly feeding behavior could contribute to lethality. Biological and biochemical differences between B. tabaci cryptic species should also be considered. The results are summarized in Table 1.

TABLE 1
Summary of the results of three replicated experiments
based on mortality post-ingestion access feeding on the
35S_Duplex transgenic line, piTop4_C7 transgenic line,
piTop4_C7_GNRA_mito2 transgenic line,
and negative control

| | | | piTop4_{—} | |
|---|---|---|---|---|
| | 35S_{—} | piTop4_{—} | C7_GNRA_{—} | |
| Parameter | Duplex | C7 | mito2 | Control |
| Mean percent mortality | ~76% | ~57% | 66% | ~14% |
| K-M significance vs | p < | p < | p < | — |
| control | 0.0001 | 0.0001 | 0.0001 | |
| Relative whitefly | +++ | + | ++ | — |
| mortality | | | | |
| Gene expression | ~1.8 | 2.08 | 2.08 | — |
| knockdown as fold- | | | | |
| change | | | | |

TABLE 2
Fold change in expression of different genes of interest
for three transgenic tomato lines

| | AGLU | TRE | TRET | C7 | D24 | Crc |
|---|---|---|---|---|---|---|
| Transgenic Line 802 | 2.15 | 1.46 | 1.58 | 1.15 | 1.28 | 3.09 |
| Transgenic Line 804 | 2.25 | 1.69 | 1.45 | 1.14 | 1.57 | 4.37 |
| Transgenic Line 805 | 2.63 | 1.63 | 1.71 | 1.51 | 2.61 | 2.41 |

### CONCLUSIONS

Dual-pathway RNAi constructs showed effective silencing of essential whitefly genes thereby causing significant mortality.
Comparable mortality was observed with the single-target, piRNA-based transgenes to the dual construct, showing the potency of RNAi induced by the pathway.
RNAi in transgenic tomato plants shows promise to be used as a tool for whitefly control and virus transmission management.
The approach may also be integrated with other IPM strategies including biological control and applying soft chemistries (insecticides) for sustainable pest and virus management.

Thus, development and efficacy of the first dual-pathway RNAi constructs, e.g. siRNA+piRNA combined, showed a significant advantage and improvement over traditional single (dsRNA) pathway RNAi.

Transgenic lines piTop4_C7, piTop4_C7_GNRA_mito2 (expressing piRNA) and 345S_Duplex_cotton (expressing a combination of si-RNA/pi-RNA) showed ~55-76% whitefly mortality respectively (Table 1), significantly higher than natural mortality. By comparison, the tomato line expressing vector only (negative control) showed ~14% mortality. Targeted knockdown of AGLU, TRE, TRET, D24, C7 and Crc was confirmed and quantified by real-time quantitative PCR results depicting a knockdown ranging from ~25-80%, with overall ~2-fold (~50%) expression down-regulation, or about 50% reduced expression (Table 2). The data provides proof of dual-RNAi knockdown and mortality post-whitefly IAP on the C7 transgene target.

In addition, GNRA tetraloops lend stability to piRNAs due to the non-standard secondary structure formation which will inhibit exonuclease activity and stabilize the RNA making it more available for endonucleases like piwi proteins thereby leading to an expedited process of piRNA generation ultimately leading to persistent expression knockdown.

“Mitomer2”, a mitochondrion binding aptamer was also added to one of the constructs (FIG. 6C). As phasing occurs on the surface of mitochondria so localization of the synthetic RNA to the organelle may promote the production of piRNAs.

### REFERENCES

Each of the following references are herein incorporated by reference in their entirety.
1. Vyas, M.; Raza, A.; Ali, M. Y.; Ashraf, M. A.; Mansoor, S.; Shahid, A. A.; Brown, J. K. Knock down of whitefly gut gene expression and mortality by orally delivered gut gene-specific dsRNAs. PLOS One 2017, 12, e0168921.
2. Arad, N.; Paredes-Montero, J. R.; Mondal, M. H.; Ponvert, N.; Brown, J. K. RNA interference mediated knockdown of genes involved in sugar transport and metabolism disrupts psyllid Bactericera cockerelli (Order: Hemiptera) gut physiology and results in high mortality. Frontiers in Insect Science 2023, 3, 1283334.
3. Mondal, M.; Brown, J. K.; Flynt, A. Exploiting somatic piRNAs in Bemisia tabaci enables novel gene silencing through RNA feeding. Life science alliance 2020, 3.
4. Malik, H. J.; Raza, A.; Amin, I.; Scheffler, J. A.; Scheffler, B. E.; Brown, J. K.; Mansoor, S. RNAi mediated mortality of the whitefly through transgenic expression of double-stranded RNA homologous to acetylcholinesterase and ecdysone receptor in tobacco plants. Scientific Reports 2016, 6, 38469.
5. Zubair, M.; Khan, M. Z.; Rauf, I.; Raza, A.; Shah, A. H.; Hassan, I.; Amin, I.; Mansoor, S. Artificial micro RNA (amiRNA)-mediated resistance against whitefly (Bemisia tabaci) targeting three genes. Crop Protection 2020, 137, 105308.
6. Shamimuzzaman, M.; Hasegawa, D. K.; Chen, W.; Simmons, A. M.; Fei, Z.; Ling, K.-S. Genomewide profiling of piRNAs in the whitefly Bemisia tabaci reveals cluster distribution and association with begomovirus transmission. PLOS One 2019, 14, e0213149.
7. Haldar, S.; Kührová, P.; Banas, P.; Spiwok, V.; Sponer, J.; Hobza, P.; Otyepka, M. Insights into stability and folding of GNRA and UNCG tetraloops revealed by microsecond molecular dynamics and well-tempered metadynamics. Journal of Chemical Theory and Computation 2015, 11, 3866-3877.
8. Tawaraya, Y.; Hyodo, M.; Ara, M. N.; Yamada, Y.; Harashima, H. RNA aptamers for targeting mitochondria using a mitochondria-based SELEX method. Biological and Pharmaceutical Bulletin 2014, 37, 1411-1415.
9. Susanta Haldar, Petra Kührová, Pavel Banáš, Vojtěch Spiwok, Jiří Šponer, Pavel Hobza, and Michal Otyepka. Insights into Stability and Folding of GNRA and UNCG Tetraloops Revealed by Microsecond Molecular Dynamics and Well-Tempered Metadynamics. Journal of Chemical Theory and Computation 2015 11 (8), 3866-3877. DOI: 10.1021/acs.jctc.5b00010.
10. Flynt, A. S., Insecticidal RNA interference, thinking beyond long dsRNA, Pest Manag.Soc., 2021, 77:2179-2187.
11. Flynt, A. S. and J. K. Brown, Application of piRNA-Triggered Gene Silencing in the Phloem-Feeding Whitefly Bemisia tabaci B mitotype NA-ME cryptic species, No. 49 Oral Presentation-Symposium, 2025 (Abstract).
12. Paredes-Montero, J. R. et al., Knockdown of ecdysteroid synthesis genes results in impaired molting and high mortality in Bactericera cockerelli (Hemiptera: Triozidae), Pest Manag. Soc., 2022, doi 10.1002/ps.6848.
13. Saberi, E. et al., Optimal dsRNA Concentration for RNA Interference in Asian Citrus Psyllid, Insects, 2024, 15 (58): 1-15; doi.org 10.3390/insects15010058.
14. Quito, K. et al., A platform for anti-pest nucleic acid engineering based on piRNA-mediated gene silencing, Entomological Soc. of America-RNAi Symposium, Portland, OR, Nov. 9-12, 2025 (Abstract of oral presentation).
15. Shamimuzzaman, Md et al., “Genome-wide profiling of piRNAs in the whitefly Bemisia tabaci reveals cluster distribution and association with begomovirus transmission”, PLOS ONE, 2019, 14 (3): e0213149.16. Gainetdinov, I. et al., “Relaxed targeting rules help PIWI proteins silence transposons”, Nature, 2023, 619:394-402.
17. Priyadarshini, M. et al., “Reprogramming the piRNA pathway for multiplexed and transgenerational gene silencing in C. elegans”, Nat. Methods, 2022, 19 (2): 187-194.

What is claimed is:

1. A synthetic RNA for silencing a gene of a target species comprising:
a piwi-interacting RNA (pi-RNA) sequence,
a target sequence targeting the gene of the target species, and at least one of a)-b):
a) one or more tetraloops appearing at at least one end of the synthetic RNA, and
b) an RNA aptamer that binds to a receptor of a mitochondria of the target species,
wherein the synthetic RNA induces silencing of the gene of the target species.

2. The synthetic RNA of claim 1, wherein the synthetic RNA is single-stranded.

3. The synthetic RNA of claim 1, wherein the synthetic RNA comprises one or more of said tetraloops at ends of the synthetic RNA, or two of said tetraloops, one at each of the 3′ and 5′ ends of the synthetic RNA.

4. The synthetic RNA of claim 3, wherein the one or more tetraloops has the sequence
(SEQ ID NO: 6)
CGCGAGAGCG.

5. The synthetic RNA of claim 1, wherein the synthetic RNA comprises the RNA aptamer.

6. The synthetic RNA of claim 5, wherein the RNA aptamer has the sequence
(SEQ ID NO: 7)
CUUAGCCCAUAGCUGGCUGC.

7. The synthetic RNA of claim 1, wherein the target species is a hemipteran organism.

8. The synthetic RNA of claim 1, wherein the hemipteran organism is a whitefly.

9. The synthetic RNA of claim 1, wherein the target species is Bemisia tabaci.

10. The synthetic RNA of claim 9, wherein the gene is aquaporin 1 (AQP1) having the amino acid sequence:
(SEQ ID NO. 14)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAG NLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF.

11. The synthetic RNA of claim 1, wherein the gene is selected from the group consisting of: aquaporin (AQP1) (SEQ ID NO. 14), alpha glucosidase 1 (AGLU1), vATPase-A, v-ATPase-B, v-APTase-D, v-ATPase-E, Delta-24 sterol reductase (D-24), cholesterol desaturase (C7) (SEQ ID NO. 5), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), and Actin related ⅔.

12. The synthetic RNA of claim 1, wherein the piwi-interacting RNA is selected from the group consisting of piTop1 (SEQ ID NO. 1), piTop2 (SEQ ID NO. 2), piTop3 (SEQ ID NO. 3), and piTop4 (SEQ ID NO. 4).

13. A RNA interference (RNAi) construct comprising the synthetic RNA of claim 1 and a small interfering RNA (siRNA).

14. A vector comprising the synthetic RNA of claim 1.

15. The vector of claim 14, wherein the vector is a transposon vector.

16. A cell comprising the synthetic RNA of claim 1.

17. A composition comprising the synthetic RNA of claim 1.

18. A kit comprising the synthetic RNA of claim 1, and the synthetic RNA for pesticide treatment, pest control, or management of pests.

19. A method of regulating the expression of a target gene in an organism, comprising providing the synthetic RNA of claim 1 to the organism, wherein the synthetic RNA hybridizes to at least a portion of a mRNA transcript of the target gene and causes downregulation or silencing of the target gene.

20. The method of claim 19, wherein the organism is a hemipteran.

21. A transgenic plant comprising the synthetic RNA of claim 1.

22. The transgenic plant of claim 21, wherein the plant is from the Solanaceae family.

23. The transgenic plant of claim 21, wherein the plant is a tomato plant.


SEQUENCE LISTING (14 sequences)

SEQ ID NO: 1 - RNA, 35 nt, synthetic construct
  source 1..35: mol_type = other RNA
  modified_base order(2..4,12..13,15,17,20,22..23,26..27,33): mod_base = OTHER; note = thymine
atttgcgcacattatctagtgttacttgagagtcc

SEQ ID NO: 2 - RNA, 58 nt, synthetic construct
  source 1..58: mol_type = other RNA
  modified_base order(2..4,12..13,15,17,20,22..23,26..27,33,38,41,44..45,47,55..57): mod_base = OTHER; note = thymine
atttgcgcacattatctagtgttacttgagagtccggtgctgattctaggaacatttc

SEQ ID NO: 3 - RNA, 98 nt, synthetic construct
  modified_base order(2..4,12..13,15,17,20,22..23,26..27,33,38,41,44..45,47,55..57,60..65,67..68,70..71,74,77,80..85,89..90,92..94,96..97): mod_base = OTHER; note = thymine
  source 1..98: mol_type = other RNA
atttgcgcacattatctagtgttacttgagagtccggtgctgattctaggaacatttcgttttttcttcttggtgctggttttttcacttctttgttc

SEQ ID NO: 4 - RNA, 150 nt, synthetic construct
  modified_base order(2..4,12..13,15,17,20,22..23,26..27,33,38,41,44..45,47,55..57,60..65,67..68,70..71,74,77,80..85,89..90,92..94,96..97,99..100,104..106,108..109,116,119,123,125,131..132,136,138,142,150): mod_base = OTHER; note = thymine
  source 1..150: mol_type = other RNA
atttgcgcacattatctagtgttacttgagagtccggtgctgattctaggaacatttcgttttttcttcttggtgctggttttttcacttctttgttcttgggtttcttggccactggtgactctggccgttgcctgtccgtcggcgagt

SEQ ID NO: 5 - RNA, 251 nt, synthetic construct
  source 1..251: mol_type = other RNA
  modified_base order(1,7..9,14..16,19..20,28,30,34,39,41..42,46,49,51,54..55,61,64,77,82,85..86,90..91,96,99..101,107..108,114,124,126,132..136,142,144,152,155,159..162,167,169..170,175,181,190,201..202,204,211,216,218,239,241..242,249): mod_base = OTHER; note = thymine
tcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatgg

SEQ ID NO: 6 - RNA, 10 nt, synthetic construct
  source 1..10: mol_type = other RNA
cgcgagagcg

SEQ ID NO: 7 - RNA, 20 nt, synthetic construct
  source 1..20: mol_type = other RNA
cttagcccatagctggctgc

SEQ ID NO: 8 - RNA, 286 nt, synthetic construct
  modified_base order(2..4,12..13,15,17,20,22..23,26..27,33,36,42..44,49..51,54..55,63,65,69,74,76..77,81,84,86,89..90,96,99,112,117,120..121,125..126,131,134..136,142..143,149,159,161,167..171,177,179,187,190,194..197,202,204..205,210,216,225,236..237,239,246,251,253,274,276..277,284): mod_base = OTHER; note = thymine
  source 1..286: mol_type = other RNA
atttgcgcacattatctagtgttacttgagagtcctcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatgg

SEQ ID NO: 9 - RNA, 309 nt, synthetic construct
  source 1..309: mol_type = other RNA
  modified_base order(2..4,12..13,15,17,20,22..23,26..27,33,38,41,44..45,47,55..57,59,65..67,72..74,77..78,86,88,92,97,99..100,104,107,109,112..113,119,122,135,140,143..144,148..149,154,157..159,165..166,172,182,184,190..194,200,202,210,213,217..220,225,227..228,233,239,248,259..260,262,269,274,276,297,299..300,307): mod_base = OTHER; note = thymine
atttgcgcacattatctagtgttacttgagagtccggtgctgattctaggaacatttctcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatgg

SEQ ID NO: 10 - RNA, 349 nt, synthetic construct
  modified_base order(2..4,12..13,15,17,20,22..23,26..27,33,38,41,44..45,47,55..57,60..65,67..68,70..71,74,77,80..85,89..90,92..94,96..97,99,105..107,112..114,117..118,126,128,132,137,139..140,144,147,149,152..153,159,162,175,180,183..184,188..189,194,197..199,205..206,212,222,224,230..234,240,242,250,253,257..260,265,267..268,273,279,288,299..300,302,309,314,316,337,339..340): mod_base = OTHER; note = thymine
  source 1..349: mol_type = other RNA
atttgcgcacattatctagtgttacttgagagtccggtgctgattctaggaacatttcgttttttcttcttggtgctggttttttcacttctttgttctcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatgg

SEQ ID NO: 11 - RNA, 401 nt, synthetic construct
  modified_base order(2..4,12..13,15,17,20,22..23,26..27,33,38,41,44..45,47,55..57,60..65,67..68,70..71,74,77,80..85,89..90,92..94,96..97,99..100,104..106,108..109,116,119,123,125,131..132,136,138,142,150..151,157..159,164..166,169..170,178,180,184,189,191..192,196,199,201,204..205,211,214,227,232,235..236,240..241,246,249..251,257..258,264,274,276,282..286,292,294,302,305,309..312,317,319..320,325,331,340,351..352,354,361,366,368,389,391..392,399): mod_base = OTHER; note = thymine
  source 1..401: mol_type = other RNA
atttgcgcacattatctagtgttacttgagagtccggtgctgattctaggaacatttcgttttttcttcttggtgctggttttttcacttctttgttcttgggtttcttggccactggtgactctggccgttgcctgtccgtcggcgagttcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatgg

SEQ ID NO: 12 - RNA, 421 nt, synthetic construct
  source 1..421: mol_type = other RNA
  modified_base order(12..14,22..23,25,27,30,32..33,36..37,43,48,51,54..55,57,65..67,70..75,77..78,80..81,84,87,90..95,99..100,102..104,106..107,109..110,114..116,118..119,126,129,133,135,141..142,146,148,152,160..161,167..169,174..176,179..180,188,190,194,199,201..202,206,209,211,214..215,221,224,237,242,245..246,250..251,256,259..261,267..268,274,284,286,292..296,302,304,312,315,319..322,327,329..330,335,341,350,361..362,364,371,376,378,399,401..402,409): mod_base = OTHER; note = thymine
cgcgagagcgatttgcgcacattatctagtgttacttgagagtccggtgctgattctaggaacatttcgttttttcttcttggtgctggttttttcacttctttgttcttgggtttcttggccactggtgactctggccgttgcctgtccgtcggcgagttcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatggcgcgagagcg

SEQ ID NO: 13 - RNA, 441 nt, synthetic construct
  source 1..441: mol_type = other RNA
  modified_base order(12..14,22..23,25,27,30,32..33,36..37,43,48,51,54..55,57,65..67,70..75,77..78,80..81,84,87,90..95,99..100,102..104,106..107,109..110,114..116,118..119,126,129,133,135,141..142,146,148,152,160..161,167..169,174..176,179..180,188,190,194,199,201..202,206,209,211,214..215,221,224,237,242,245..246,250..251,256,259..261,267..268,274,284,286,292..296,302,304,312,315,319..322,327,329..330,335,341,350,361..362,364,371,376,378,399,401..402,409): mod_base = OTHER; note = thymine
cgcgagagcgatttgcgcacattatctagtgttacttgagagtccggtgctgattctaggaacatttcgttttttcttcttggtgctggttttttcacttctttgttcttgggtttcttggccactggtgactctggccgttgcctgtccgtcggcgagttcgccatttgagatttgcttcacgggctgtggctcccatgttgggtcctctccttcagcatgataccagacaaaaatgagatcgttcacttcacatgatttccaagttcggactcgcgcaaagtctggaactttttcagaatatggcacgctggtgcattttccactattcccatcgaactgccagccgtggaaaggacattctaagcagtcacctctgaccaccccaccaagacccatgttggcccatggcttagcccatagctggctgccgcgagagcg

SEQ ID NO: 14 - protein, 262 aa, synthetic construct
MEDISSSGEEISMKAISKVIGVPDIRDGPTLTKCIVAEFVGTLLLVLIGCMSVAFVHQDNFVDVVKIAMAFGLIIASMVQAIGHVSGCHINPAVTCGLAVSGHVSIIKGMLYIVAQCLGAICGAIILNEITPKTGYTAAGNLGVTTLSTGVSDLQGVAIEALITFVLLLVVQSVCDGKRTDIKGSIGVAIGFAIACCHLAAIKYTGASMNPARSLGPAFVSGIWDKHWVYWAGPILGGVTASLLYAITFKAKKRSDESSYDF